(19) United States
(12) Patent Application Publication
(10) Pub. No.: US 2023/0028918 A1
(43) Pub. Date: Jan. 26, 2023

(54) COMPOSITIONS COMPRISING PKC-BETA INHIBITORS AND PROCESSES FOR THE PREPARATION THEREOF

(71) Applicant: MINGSIGHT PHARMACEUTICALS, INC., San Diego, CA (US)

(72) Inventors: Michael NIESMAN, San Diego, CA (US); Kai ZHANG, Irmo, SC (US); Kay OLMSTEAD, Escondido, CA (US)

(21) Appl. No.: 17/782,091

(22) PCT Filed: Dec. 4, 2020
(86) PCT No.: PCT/US2020/063480

§ 371 Date: Jun. 2, 2022

Related U.S. Application Data
(60) Provisional application No. 62/944,699, filed on Dec. 6, 2019.

(51) Int. Cl. A61K 31/506; A61K 31/519; A61K 45/06

(52) U.S. Cl. CPC A61K 31/506; A61K 31/519; A61K 45/06

(57) ABSTRACT

The present invention relates, in some respects, to methods of using, and compositions comprising, a protein kinase inhibitor and pharmaceutically acceptable salts, solvates, and hydrates thereof. In some embodiments, the present invention relates to modified or extended release pharmaceutical formulations in the form of particles which, in some embodiments, are used in a tablet, capsule, or particulate form, for slowly releasing the protein kinase inhibitor, or a pharmaceutically acceptable salt, solvate, or hydrate thereof, over periods of time from at least 8 to 12 hours. The compositions of the present invention are useful in the treatment of PKCβ related disorders.

### CROSS-REFERENCE TO RELATED APPLICATIONS

This application claims the benefit of U.S. Provisional Patent Application No. 62/944,699 filed on Dec. 6, 2019, which is herein incorporated by reference in its entirety.

### BACKGROUND OF THE INVENTION

A need exists in the therapeutic and medicinal art for compositions and methods of using said compositions for the effective treatment of cancer.

### BRIEF SUMMARY OF THE INVENTION

The present invention relates, in some embodiments, to methods of using, and compositions comprising a protein kinase inhibitor compound and pharmaceutically acceptable salts, solvates, and hydrates thereof. In some embodiments, the present invention relates to modified or extended release pharmaceutical formulations, preferably in the form of particles which are used in a tablet, capsule, or particulate form, for slowly releasing the protein kinase inhibitor, or a pharmaceutically acceptable salt, solvate, or hydrate thereof, over periods of time from at least 8 to 12 hours. In some embodiments, the modified or extended release pharmaceutical formulations contains both an immediate release formulation, as well as an extended release formulation. In other embodiments, the modified release pharmaceutical formulations comprise just the extended release formulation. The invention also relates to a method for preparing the extended release formulations. In some embodiments, the protein kinase inhibitor compound is a protein kinase C beta inhibitor. In some embodiments, the protein kinase inhibitor compound is 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt, solvate, or hydrate thereof.

The compositions of the present invention are useful in the treatment of, for example: cancer, such as β-cell malignancies including CLL or SLL; autoimmune disorders, such as rheumatoid arthritis, multiple sclerosis, inflammatory bowel disease, Crohn's disease, or encephalitis; or inflammation, such as inflammation caused by inflammatory bowel disease, Crohn's disease, or ulcerative colitis.

### BRIEF DESCRIPTION OF THE DRAWINGS

FIG. 1A and FIG. 1B shows that Compound A inhibits phosphorylation of PKCβ and downstream targets. Inhibition of BCR signaling in primary CLL cells treated with compound A is demonstrated. In FIG. 1A, a representative immunoblot shows decreased phosphorylation of PKCβ and its downstreamtargets with compound A. In FIG. 1B, immunoblots were quantified (pPKCβ: n=5, pERK: n=4, pIκBα: n=5, pGSK3β: n=5) using Alphaview SA software and results are reported as fold change in expression compared to vehicle control.

FIG. 2 shows inhibition of pro-inflammatory cytokine expression by primary CLL cells treated with compound A. Primary CLL cells were treated with 5 μM compound A in the presence or absence of anti-IgM ligation for 24 hrs. CCL3 and CCL4 secretion was measured by ELISA.

FIG. 3A and FIG. 3B shows that compound A reduces activation of primary CLL cells with WT and C481S BTK. Activiation of primary CLL cells treated with compound A pre- and post-ibrutinib was observed. Cryopreserved baseline samples and post-relapse samples from ibrutinib treated patients (n=2) were thawed and treated with up to 10 μM compound A and stimulated with 3.2 M CpG. CD86 (FIG. 3A) and HLA-DR (FIG. 3B) expression was measured by flow cytometry at 48 hrs. Mean fluorescence intensity (MFI) is reported. Error bars represent standard deviation.

FIG. 4 shows the effects of compound A on healthy T cells. Healthy donor T cells were treated with 1 μM compound A (n=9) and stimulated with 10 μg plate-bound anti-CD3 and 1 μg soluble anti-CD28 for 24 hours. TNFα expression was measured by ELISA.

FIG. 5 shows compound A inhibits PKCβ function in vivo and inhibits phosphorylation of SERBP1 in vivo. A phosphor-flow assay was employed to measure the phosphorylation of SERBP1, a novel substrate for PKCP. Whole blood samples were taken at the appropriate time points from CLL patients receiving compound A as part of a phase 1 study, and were shipped overnight and processed for testing the next day. Whole blood was stimulated with PMA+ionomycin, cells were permeabilized, and the amount of SERBP1 phophorylation was measured. The reported data are the CD19+pSERBP1+ population, normalized to each patient's own unstimulated sample at the corresponding time point.

FIG. 6 shows provides a summary of compound A biological activity.

FIG. 7 shows the PK/PD Data Correlation Table for patients dosed with compound A, and quantifies the increase in SERBP1 phosphorylation after PMA stimulation vs. compound A drug concentration.

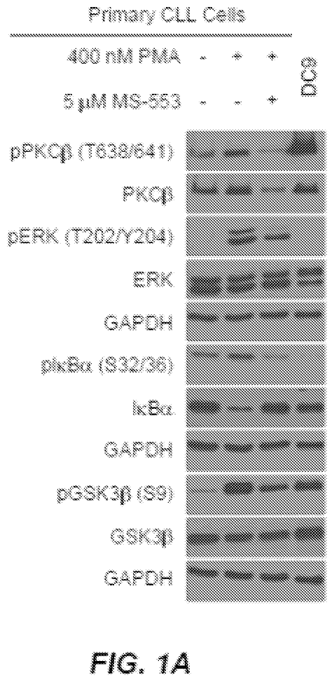

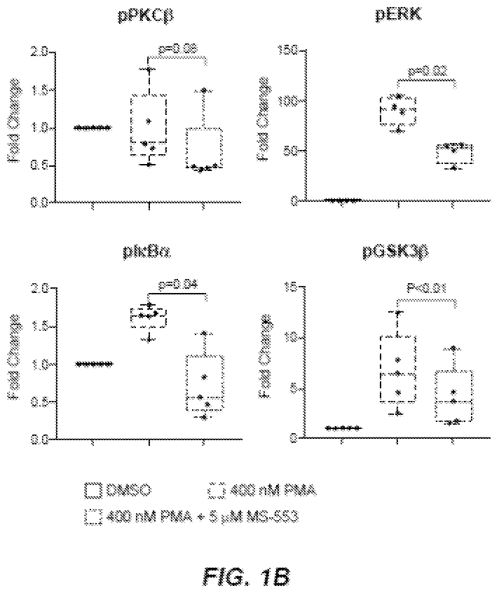

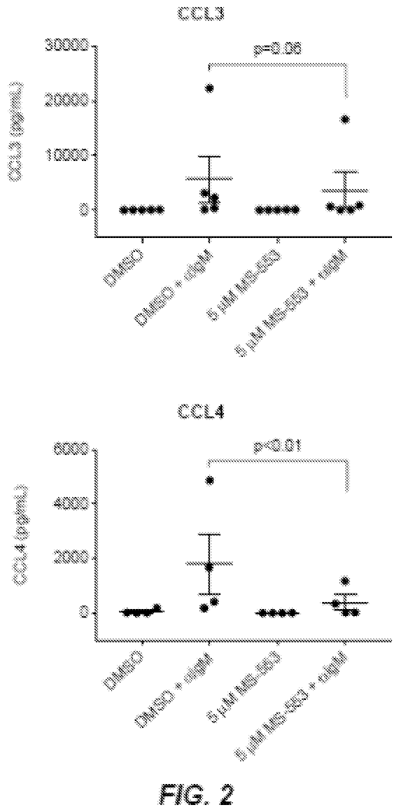

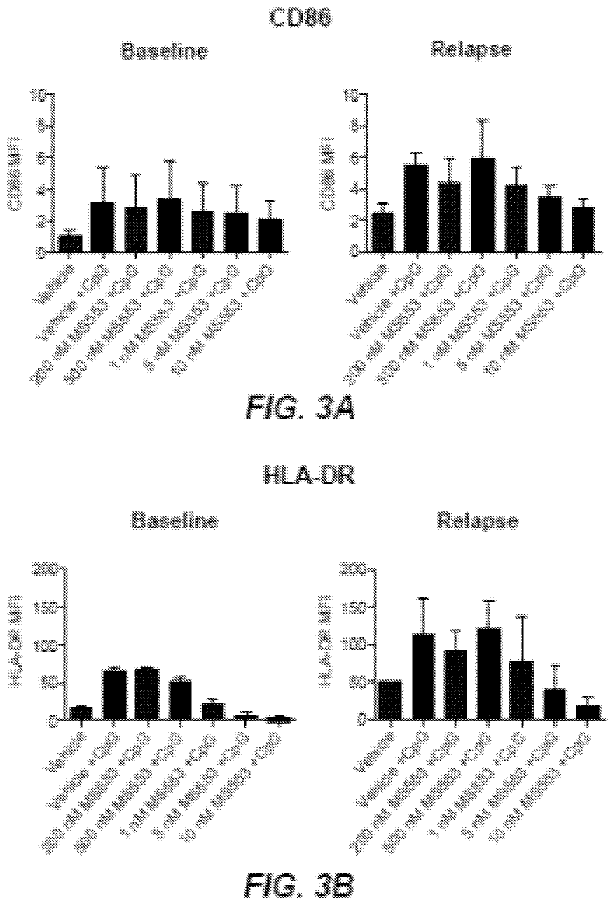

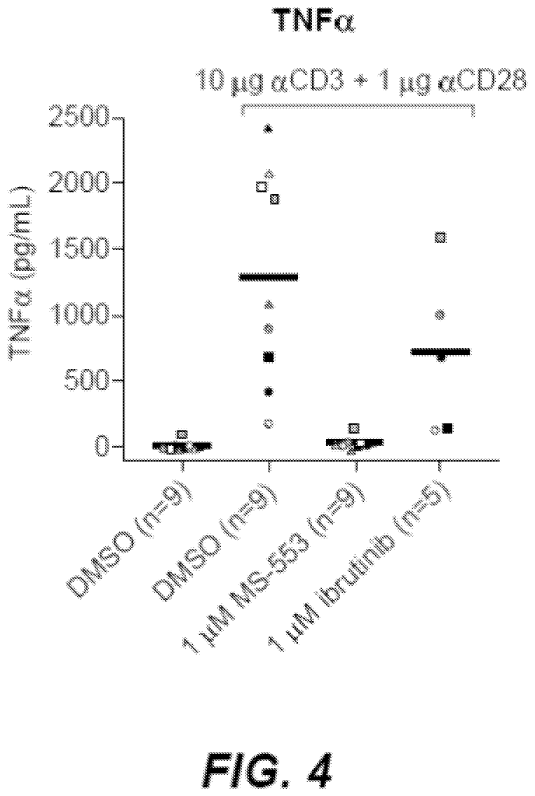

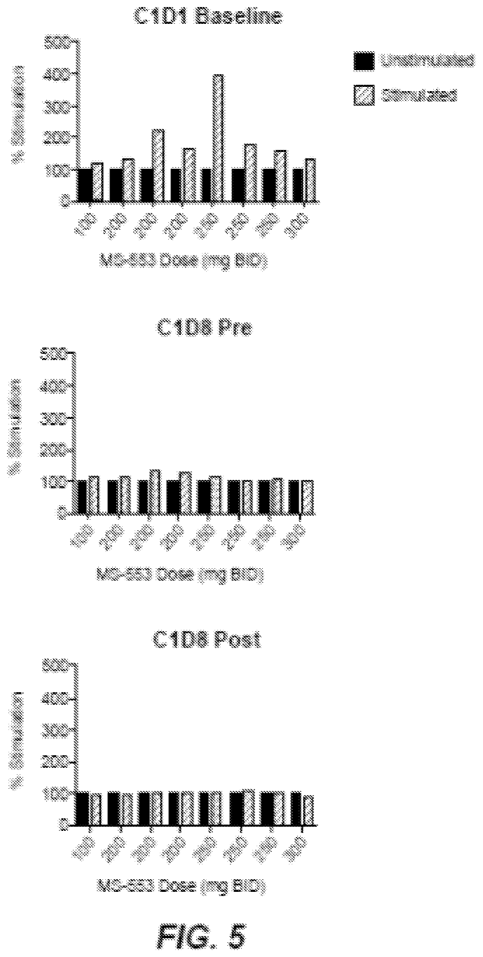

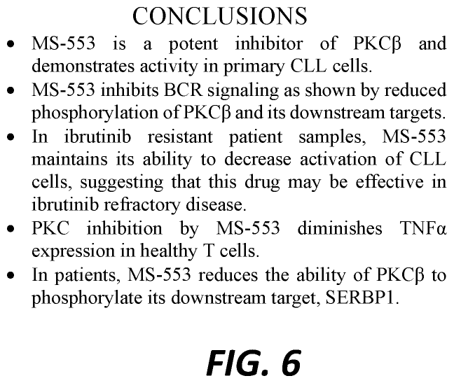

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

For clarity and consistency, the following definitions will be used throughout this patent document.

The term “inhibitor” as used herein refers to a moiety that interacts with and inactivates a protein kinase, for instance PKC0, and can thereby initiate a physiological or pharmacological response characteristic of that enzyme.

The term “in need of treatment” and the term “in need thereof” when referring to treatment are used interchangeably and refer to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” refers to any animal, including mammals, preferably mice, rats, other rodents, rabbits, dogs, cats, swine, cattle, sheep, horses, or primates, and most preferably humans.

The term “modulate or modulating” refers to an increase or decrease in the amount, quality, response or effect of a particular activity, function or molecule.

The term “composition” refers to a compound, including but not limited to, salts, solvates, and hydrates of a compound of the present invention, in combination with at least one additional component.

The term “pharmaceutical composition” refers to a composition comprising at least one active ingredient, such as a compound as described herein; including but not limited to, salts, solvates, and hydrates of compounds of the present invention, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “hydroxypropyl methylcellulose” (HPMC), which may be also referred to as “hypromellose”, refers to a propylene glycol ether of methylcellulose. The hydroxypropyl methylcellulose is available in varying degrees of viscosity. As an example, the hydroxypropyl methylcellulose may be a hydroxypropyl methylcellulose having a viscosity of about 2300 mPA seconds to about 3800 mPA seconds when present in an amount of about 2% in water at 20° C. As an example, the hydroxypropyl methylcellulose may be Methocel™ K4M Premium CR. As an example, the hydroxypropyl methylcellulose may be a hydroxypropyl methylcellulose having a viscosity of about 75 mPA seconds to about 120 mPA seconds when present in an amount of about 2% in water at 20° C. As an example, the hydroxypropyl methylcellulose may be Methocel™ K100 Premium LVCR. As another example, the hydroxypropyl methylcellulose may be Methocel™ K100M.

The term “Eudragit®” refers to a family of targeted drug release coating polymers. These polymers allow drugs to be formulated in enteric, protective or sustained-release formulations to prevent break-down of the drug until it has reached an area with adequate pH in the gastrointestinal (GI) tract. Once the drug reaches its target area of the gastrointestinal tract (i.e., duodenum, stomach) it will release from the polymer matrix and be absorbed. Targeted drug release is often used to prevent dissolution of a drug in an area where the pH is not adequate for absorption, or to help minimize gastrointestinal tract irritation. Eudragit® RLPO is a copolymer of ethyl acrylate, methyl methacrylate, and trimethylammonioethyl methacrylate chloride with the ratio 1:2:0.2. The copolymer is insoluble, has high permeability, and pH dependent swelling, making it a good candidate for sustained release tablet formulations.

The term “ethylcellulose” refers to a polymer of ethylcellulose. Ethocel™ products are water-insoluble polymers approved for global pharmaceutical applications and used in extended release solid dosage formulations. Ethocel™ is colorless, odorless, tastless, and non-caloric. Ethocel™ has been used in the pharmaceutical industry as a tablet coating, controlled-release coating, microencapsulation, and taste masking.

The term “Carbopol®” refers to a family of polymers of polyacrylic acid. Carbopol® polymers are generally high molecular weight, crosslinked polyacrylic acid polymers. Carbopol® 71G NF polymer was designed for use in oral solid dose applications. Carbopol 71G NF polymer is a free-flowing granular form of the polyacrylic acid homopolymer. Typical usage levels for achieving extended release characteristics in tablets manufactured by direct compression are 10-30 wt. %, depending on the drug properties and co-excipients.

The term “hydroxypropylcellulose” (HPC) refers to a propylene glycol ether of cellulose. HPC is a nonionic water-soluble cellulose ether formed from cellulose and propylene oxide. It combines solubility in aqueous and polar organic solvents, thermoplasticity, and surface activity with the thickening and stabilizing properties of other water-soluble cellulose polymers. Klucel™ HF Pharma is a high molecular weight (1,150,000) pharmaceutical grade of hydroxypropylcellulose with a viscosity range of 1,500-3,000 cps. Klucel™ HXF Pharma is the fine particle size of the Klucel™ HF Pharma.

The term “Methocel™ Cellulose Ethers” refers to a family of copolymers of methylcellulose and hydroxypropyl methylcellulose. Methocel™ Cellulose Ethers are water-soluble polymers. The Methocel™ polymers encompasses methylcellulose and hydroxypropyl methylcellulose (hypromellose)—each available in different grades, physical forms and a wide range of viscosities. They enable formulators to create reliable formulas for tablet coatings, granulation, controlled release, extrusion, molding and for controlled viscosity in liquid formulations. Methocel™ E (hypromellose 2910 USP) and K (hypromellose 2208, USP) are the most widely used grades in matrix formulations. The USP code is based on the substitution of the cellulose. The first two digits represent the mean % methoxyl substitution and the second two digits represent the mean % hydroxypropyl substitution. HPMC is highly hydrophilic, hydrating rapidly in contact with water. Since the hydroxypropyl group is hydrophilic and the methoxyl group is hydrophobic, the ratio of hydroxypropyl to methoxyl content influences drug release.

The term “cured composition” as used herein refers to a pharmaceutical composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A) or pharmaceutically acceptable salts, solvates, and hydrates thereof, the first excipient, and the second excipient that are cured together.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver or by an individual, which includes one or more of the following:

(1) preventing the disease, for example, preventing a disease, condition or disorder in an individual that may be predisposed to the disease, condition or disorder but does not yet experience or display the pathology or symptomatology of the disease;

(2) inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) ameliorating the disease, for example, ameliorating a disease, condition or disorder in an individual that is experiencing or displaying the pathology or symptomatology of the disease, condition or disorder (i.e., reversing the pathology and/or symptomatology).

The term “an amount equivalent to”, followed by the recitation of an amount of Compound A (such as, for example 0.01 mg of Compound A) refers to the amount of 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A) or pharmaceutically acceptable salts, solvates, and hydrates thereof that is equivalent to the recited amount of Compound A.

The term “% by weight”, when referring to an amount of a component that is present in a composition—such as Compound A, or such as an excipient—refers to the amount of that component as a % by weight of the composition.

The term “release rate”, also referred to as a “dissolution rate” herein, with reference to a compound, refers to the percentage amount of that compound that is released in an aqueous medium over a specified time period. As an example, the recitation “a release rate by weight of the compound in an aqueous medium that is release rate (a), wherein (a) about 15% to about 35% by weight of the compound is released over the first two hours” means that the percentage by weight of the compound that is released over the first two hours is about 15% to about 35% by weight of the initial amount of the compound. The term “release profile”, also referred to as a “dissolution profile” herein, with reference to a compound, refers to a plot showing the percentage amount of that compound that is released in an aqueous medium overtime. The aqueous medium may be an aqueous medium as described herein.

### Protein Kinase Inhibitors

Compounds that are kinase inhibitors have the potential to provide therapeutically effective pharmaceutical compositions that would be expected to have beneficial and improved pharmaceutical properties for the treatment of kinase related conditions or disorders such as cancer and other proliferative disorders.

Discussed herein is 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine and referred to herein as Compound A or MS-553. Compound A has been previously described in WO 2008/096260 and related patents and patent applications, e.g. U.S. Pat. No. 8,183,255, and U.S. patent application Ser. No. 14/506,470, each of which is incorporated by reference in their entirety.

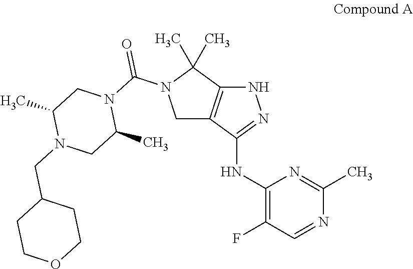 [SMILES: Cc1ncc(F)c(Nc2n[nH]c3c2CN(C(=O)N2C[C@@H](C)N(CC4CCOCC4)C[C@@H]2C)C3(C)C)n1]

### 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine

A summary of the Protein Kinase C (PKC) inhibition by Compound A is provided in Table 1. The methods for these determinations have been described (Grant, et al. 2010, Eur. J. Pharmacol. 627:16-25). Compound A is a potent, ATP-competitive, and reversible inhibitor of conventional PKC enzymes with a Kᵢ=5.3 nM for recombinant PKC beta and a Kᵢ=10.4 nM for recombinant PKC alpha. It also is a potent inhibitor of the novel isoform PKC theta with an IC₅₀=25.6 nM. Furthermore, it demonstrated some potency for conventional isoform PKC gamma with an IC₅₀=57.5 nM. Otherwise, it demonstrates a high degree of selectivity for the other members of the conventional, novel and atypical isoforms of PKC as shown by lower potency against these isoforms (Table 1). Compound A does not significantly inhibit PKC delta.

TABLE 1

| In Vitro Assays | IC₅₀ (nM) | Ki (nM) |
|---|---|---|
| Human PKC alpha | | 10.4 |
| Human PKC betaII | | 5.3 |
| Human PKC alpha | 2.3 | |
| Human PKC betaI | 8.1 | |
| Human PKC betaII | 7.6 | |
| Human PKC theta | 25.6 | |
| Human PKC gamma | 57.5 | |
| Human PKC mu | 314 | |
| Human PKC epsilon | 808 | |
| Human PKC delta | >1000 | |
| Human PKC eta | >1000 | |
| Human PKC iota | >1000 | |
| Human PKC zeta | >1000 | |
| Human PRKCN (PKD3) | 131 | |
| pSHP2 (PKCβ cell assay) | 9.8 | |
| Interleukin-8 release | 39 | |

As a selective inhibitor of PKC, Compound A is useful in the treatment of conditions in which PKC has demonstrated a role in the pathology, such as cancer, immune disorders and inflammation, through inhibition of PKCβ signaling. Clinical testing of BTK inhibitors, however, have demonstrated that the near 100% inhibition of the B-cell receptor (BCR) to NFκB signaling pathway is critical for efficacy in oncology indications and especially B-cell mediated diseases. Thus, a critical aspect in the development of Compound A as a useful therapy for such diseases and disorders is the development of modified or extended release formulations designed to maintain 100% inhibition of the pathway (e.g., through 100% inhibition of PKCβ signaling) while maintaining Cₘₐₓ values as low as possible to limit possible adverse events.

Provided herein are extended release (ER) formulations of Compound A that are developed to control the release of Compound A. Delayed release allows for plasma drug concentrations to be maintained at a high enough level to inhibit PKCβ signaling for a longer period of time than is possible with instantaneous or immediate release (IR) formulations. The ER formulations thus require less frequent dosing in order to maintain therapeutic drug concentrations.

As shown in Example 1, biomarker data from a PKCβ signaling assay performed on whole blood samples from patients with CLL or SLL suggests that concentrations of Compound A in the plasma in the range of 500-600 ng/mL completely suppress PKCβ signaling. The ability to maintain a high level of inhibition is quite important in CLL and other oncology conditions that seek to disrupt signaling via the B-cell receptor (BCR) to NFκB signaling pathway.

Additionally, clinical trial data with Compound A shows that plasma Cₘₐₓ values around 2000 ng/mL are well-tolerated when the compound is taken with food. Furthermore, food does not have a significant effect on the PK prolife of Compound A.

For oncology indications, especially B-cell mediated diseases (where clinical testing of BTK inhibitors have demonstrated that the near 100% inhibition of the pathway is critical for efficacy), an extended release formulation which maintains a Cₘᵢₙ plasma value of at least 500-600 ng/mL at 24 h after a single dose, and a Cₘₐₓ of at most 2500-3000 ng/mL is preferred. A formulation with these properties would allow for once a day dosing of the drug compound.

### Extended Release Formulations

It is of significant advantage to both the patient and clinician that medication be formulated so that it may be administered in a minimum number of daily doses from which the drug is uniformly released over a desired, extended period of time. Various techniques have been developed for the purpose of including a pharmaceutical preparation comprising a drug-containing particle with a coating layer and a pharmaceutical preparation comprising a continuous matrix with a drug dispersed therein, such as embedded into a rigid lattice of resinous material.

Various excipients, matrices, and formulations have been used to achieve the extended release of a drug substance. As disclosed herein, Compound A can be formulated using three main approaches to extend the release profile ofthe drug substance:
(1) hydrophobic matrix tablets with erosion control;
(2) hydrophilic matrix tablets with diffusion control; and
(3) controlled release coating technology.

By using such extended release formulations, efficacious blood plasma levels are maintained over at least 8-12 hours, and up to 24 hours. In some embodiments, the efficacious blood plasma level of Compound A is about 500-600 ng/mL. In some embodiments, the efficacious blood plasma level of Compound A is at least 500 ng/mL. In some embodiments, the efficacious blood plasma level of Compound A is at least 600 ng/mL. In some embodiments, the efficacious blood plasma level of Compound A is at least 700 ng/mL. In some embodiments, the efficacious blood plasma level of Compound A is about 800 ng/mL. In some embodiments, the efficacious blood plasma level of Compound A is at least 800 ng/mL. In some embodiments, efficacious blood plasma levels are maintained for at least 8 hours. In some embodiments, efficacious blood plasma levels are maintained for at least 10 hours. In some embodiments, efficacious blood plasma levels are maintained for at least 12 hours. In some embodiments, efficacious blood plasma levels are maintained for at least 18 hours. In some embodiments, efficacious blood plasma levels are maintained for up to 24 hours.

1. Hydrophobic Matrix Tablets with Erosion Control

Provided herein are hydrophobic matrix tablets wherein Compound A is mixed with a hydrophobic polymer. This causes extended release because the drug, Compound A, after being dissolved, will have to be released by diffusion through channels within the hydrophobic polymer. In these pharmaceutical preparations, the coating layer or matrix comprises a substance insoluble, or hardly soluble, in aqueous body fluids, and the release of the drug, e.g., Compound A, is controlled by means of the resistance of said coating layer or matrix against the diffusion of the drug therethrough. Such pharmaceutical preparations are characterized in that the particles used in making the matrix, are made as minimally disintegrable as possible. The release of the drug from such pharmaceutical preparations is driven by the gradient of the drug concentration resulting from penetration of water by diffusion into the formulation. In this mode of release, at the latter stage of release, the rate of the release is described by Fick's law, that is, the rate of release decreases due to the decrease in the concentration gradient and the increase in the distance of the diffusion.

In some embodiments provided herein, Compound A is formulated as described in U.S. Pat. No. 3,458,622, which discloses a controlled release tablet for the administration of medicinal agents over a prolonged period of up to about 8 hours. In some embodiments, a compressed tablet for the prolonged release of Compound A, is made up of a tablet containing Compound A in a core formed from a polymeric vinyl pyrrolidone, preferably polyvinyl pyrrolidone (PVP), and a carboxyvinyl hydrophilic polymer (hydrocolloid). In some embodiments, this core material formed from the two polymeric substances provides the controlled release effect by forming a complex under the action of water or gastric fluid.

2. Hydrophilic Matrix Tablets with Diffusion Control

Also provided herein, are hydrophilic matrix tablets wherein Compound A is mixed with a gelling agent) in which the drug is dissolved/dispersed. The drug, e.g., Compound A, is usually dispersed within a polymer and then released by undergoing diffusion. Diffusion systems rate release is dependent on the rate at which the drug dissolves through the polymer barrier. However, to make the drug extended release in this device, the rate of dissolution of the drug within the matrix is higher than the rate at which it is released. These formulations have relatively low cost and broad regulatory acceptance. The polymers used can be broken down into categories: cellulose derivatives, non-cellulose natural, and polymers of acrylic acid.

In some embodiments provided herein, Compound A is formulated as described in U.S. Pat. No. 4,140,755, which discloses sustained release tablets. In some embodiments, the sustained release tablets which contain a homogeneous mixture of Compound A with one or more hydrophilic hydrocolloids, such as hydroxypropylmethylcellulose having a viscosity of 4000 cps. In some embodiments, the hydrocolloids when contacted with gastric fluid at body temperatures form a sustained gelatinous mix on the surface of the tablet causing the tablet to enlarge and acquire a bulk density of less than 1. In some embodiments, Compound A is slowly released from the surface of the gelatinous mix which remains buoyant in the gastric fluid.

In other embodiments provided herein, Compound A is formulated as described in U.S. Pat. No. 4,259,314, which discloses a controlled long-acting dry pharmaceutical compositions. In some embodiments, the controlled long-acting dry pharmaceutical composition of Compound A includes a dry carrier formed from a mixture of hydroxypropylmethylcellulose (HPMC) (viscosity of 50 to 4000 cp in 2% aqueous solution at 20° C.) and hydroxypropylcellulose (HPC) (viscosity of 4000 to 6500 cp for a 2% aqueous solution at 25° C.).

In some embodiments provided herein, the polymer is incorporated within the tablet formulation. In some embodiments, upon exposure to an aqueous media, the presence of the polymer causes a tablet to rapidly gel and swell; and the drug, e.g., Compound A is gradually be released as it diffuses through the gel layer of the tablet and as the tablet erodes while the gel layer ingresses further into the tablet. In some embodiments, release of an insoluble drug, e.g., Compound A, is mediated primarily through tablet erosion.

In some embodiments, the rate of release of the drug, e.g., Compound A, from the matrix tablet formulation depends on:
a. the type of polymer itself,
b. the specific grade of polymer;
c. the level of polymer used;
d. the solubility of the drug, e.g. Compound A;
e. the selection of tablet excipients (soluble vs insoluble) and their levels;
f. the size of the tablet;
g. the shape of the tablet;
   and
h. combinations thereof.

Common hydrophilic polymers include, for example, PolyOx™ N60K, Carbopol® 71G, Methocel™ K100 LV, and the like.

3. Tablets with a Controlled Release Coating

Also provided herein, are matrix tablets wherein the tablet is covered with a slow-dissolving coating. The tablet will slowly release the drug, e.g., Compound A, as the coating is dissolved. Such dissolution systems are often used for compounds with high to medium solubility in water. Instead of diffusion, the drug release depends on the solubility and thickness of the coating. Because of this mechanism, the dissolution will be the rate limiting factor here for drug release.

Dissolution tablet systems can be broken down to subcategories called reservoir devices and matrix devices, based on where the drug, e.g., Compound A, is located. In a reservoir device system, as disclosed herein, a coating covers the drug with an appropriate material which will dissolve slowly (as described above). Alternatively, also provided herein are matrix tablets which have Compound A in a matrix and the matrix is dissolved slowly instead of a coating.

In some embodiments provided herein, Compound A is formulated as described in U.S. Pat. No. 4,252,786. In some embodiments, a rupturable, relatively water-insoluble, water-permeable film formed of a combination of hydrophobic and hydrophilic polymers is employed over an insoluble swelling type delayed release matrix or core containing Compound A. In some embodiments, the core includes a blend of polyvinyl pyrrolidone and a carboxyvinyl hydrophilic polymer.

In other embodiments provided herein, Compound A is formulated as described in U.S. Pat. Nos. 4,309,404 and 4,248,857, which disclose slow release tablet formulations with a slow release coating. In some embodiments, the slow release tablets are formed of a core material containing Compound A (31-53%), carboxypolymethylene (7-14.5%), zinc oxide (0-3%), stearic acid (4.5-10%), and mannitol (3-30%); a seal coating surrounding the core; and a sugar coating surrounding the seal coating. In some embodiments, the tablet formulations provide substantially zero order release of the core contained drug, e.g., Compound A, for about 12 hours following the first hour of administration. Thus, zero order release is only obtained after the initial surge of release of drug, e.g., Compound A, in the first hour.

4. Combination Controlled Release Tablets

In other embodiments provided herein, Compound A is formulated as described in U.S. Pat. No. 4,610,870, which discloses a controlled release pharmaceutical formulation which approaches zero order release of active drug. In some embodiments, the controlled release pharmaceutical formulation is a coated tablet, containing a core portion from which Compound A is slowly released over a controlled length of time. The core also includes one or more hydrocolloid gelling agents having a viscosity of within the range of from about 10,000 to about 200,000 centipoises in 2% solution at 20° C., such as hydroxypropylmethylcellulose and/or methylcellulose, one or more non-swellable binders and/or wax binders (where Compound A and/or hydrocolloid gelling agents are non-compressible), one or more inert fillers or excipients, one or more lubricants, and optionally one or more antiadherents such as dioxide and water.

In other embodiments provided herein, Compound A is formulated as described in U.S. Pat. No. 4,309,405, which discloses a sustained release tablet similar to that disclosed in U.S. Pat. No. 4,304,404 described above. In some embodiments, the core contains 20 to 70% Compound A, 30 to 72% of a mixture of a water-soluble polymer such as hydroxypropylmethylcellulose or hydroxypropylcellulose and water-insoluble polymer (ethylcellulose alone or in admixture with carboxypolymethylene, hydroxypropylcellulose and the like). In some embodiments, the tablet formulations provide substantially zero order release of the core contained drug, e.g., Compound A, for about 12 hours following the first hour of administration. Thus, zero order release is only obtained after the initial surge of release of drug, e.g., Compound A, in the first hour.

5. Compound A Tablet Formulations

Provided herein are extended release formulations for Compound A which are capable of approaching zero order or pseudo-zero release of Compound A over an 8 hour to at least a 12 hour period. In some embodiments, the release of Compound A is over at least 8 hours. In some embodiments, the release of Compound A is over at least 12 hours. In some embodiments, the release of Compound A is over at least 12 hours.

The extended release pharmaceutical formulations of the present invention comprise from 5% to 70% of Compound A within a tablet core that is either uncoated controlled release matrix tablets or a tablet core coated with a controlled release coating system.

The matrix tablet containing hydrophilic, hydrophobic, or combination of the release controlling polymeric system may also contain at least one binder, filler, glidant, and lubricant for tablet formulation, but does not contain any disintegrant in order to avoid weakening of mechanical strength upon ingestion. In some embodiments, the at least one binder, filler, glidant, and lubricant for tablet formulation is selected from the group consisting of (1) ethers of cellulose, such as hydroxypropyl methylcellulose, (2) esters of cellulose, (3) cellulose acetate, (4) ethyl cellulose, (5) polyvinyl acetate, (6) neutral copolymers based on ethylacrylate and methylmethacrylate, (7) copolymers of acrylic and methacrylic acid esters with quaternary ammonium groups, (8) pH-insensitive amino methacrylic acid copolymers, (9) polyethylene oxides, (10) polyvinylpyrrolidone, (11) polysaccharides of natural origin such as xanthan gum and locust bean gum, (12) polyethylene glycol, (13) polypropylene glycol, (14) castor oil, (15) triacetin, (16) tributyl citrate, (17) tri-ethyl citrate, (18) acetyl tri-n-butyl citrate, (19) diethyl phthalate, (20) dibutyl sebacate, (21) acetylated mono- and di-glycerides, and mixtures thereof.

In some embodiments, the formulations of the present invention are composed of a mixture of 5% to 70% of Compound A in a hydrophilic matrix tablet containing one or more hydrophilic release controlling polymers, including but not limiting to, hydroxypropyl methyl cellulose or HPMC (such as Methocel™, various molecular weight), hydroxypropylcellulose or HPC (such as Klucel™ with various molecular weight), poly(ethylene) Oxide (such as PolyOx™), soluble polyvinylpyrrolidone or Povidon (such as Kollidone® of various grades), cross-linked polyacrylic acid polymer (Carbopol®), or others. In some embodiments, the amount of Compound A is from 10% to 50%. In some embodiments, the amount of Compound A is from 35% to 45%. In some embodiments, the amount of Compound A is about 5%, about 10%, about 15%, about 20%, about 25%, about 30%, about 35%, about 40%, about 45%, about 50%, about 55%, about 60%, about 65%, or about 70%. In some embodiments, the amount of Compound A is about 40%.

In other embodiments, formulations of the present invention are composed of a mixture of 5% to 70% of Compound A in a hydrophobic matrix tablet containing one or more water insoluble release controlling polymers such including but not limiting to ethylcellulose (such as, Ethocel™), hypromellose acetate succinate, cellulose acetate, cellulose acetate propionate, Eudogrit®, and natural wax, or others. In some embodiments, the amount of Compound A is from 10% to 50%. In some embodiments, the amount of Compound A is from 35% to 45%. In some embodiments, the amount of Compound A is about 5%, about 10%, about 15%, about 20%, about 25%, about 30%, about 35%, about 40%, about 45%, about 50%, about 55%, about 60%, about 65%, or about 70%. In some embodiments, the amount of Compound A is about 40%.

In other embodiments, formulations of the present invention are composed of a mixture of 5% to 70% of Compound A in a single matrix containing a combination of hydrophilic and hydrophobic polymers. In some embodiments, the hydrophilic release controlling polymer is hydroxypropyl methyl cellulose or HPMC (such as Methocel™, various molecular weight), hydroxypropylcellulose or HPC (such as Klucel™ with various molecular weight), Poly(ethylene) Oxide (such as PolyOx™), soluble polyvinylpyrrolidone or Povidon (such as Kollidone® of various grades), cross-linked polyacrylic acid polymer (Carbopol®), or others. In some embodiments, the hydrophobic release controlling polymer is ethylcellulose (such as, Ethocel™), hypromellose acetate succinate, cellulose acetate, cellulose acetate propionate, Eudogrit®, and natural wax, or others. In some embodiments, the amount of Compound A is from 10% to 50%. In some embodiments, the amount of Compound A is from 35% to 45%. In some embodiments, the amount of Compound A is about 5%, about 10%, about 15%, about 20%, about 25%, about 30%, about 35%, about 40%, about 45%, about 50%, about 55%, about 60%, about 65%, or about 70%. In some embodiments, the amount of Compound A is about 40%.

In other embodiments, formulations of the present invention are composed of an immediate release tablet core containing 5% to 70% of Compound A which is coated with a dissolution modifying coating system. In some embodiments, the dissolution modifying system contains a pore former, in 5% to 30% weight of water insoluble coating material, in order to controls the release rate of Compound A. In other embodiments, the dissolution modifying system is physical wherein a laser or other method is used to create a hole in a coated tablets that rely on osmosis to drive drug release.

In some embodiments, the amount of Compound A is from 10% to 50%. In some embodiments, the amount of Compound A is from 35% to 45%. In some embodiments, the amount of Compound A is about 5%, about 10%, about 15%, about 20%, about 25%, about 30%, about 35%, about 40%, about 45%, about 50%, about 55%, about 60%, about 65%, or about 70%. In some embodiments, the amount of Compound A is about 40%.

In some embodiments, the extended release formulations of Compound A described herein preferably release more than 70% of drug over 24 hour period. In some embodiments, more than 70% of Compound A is released over a 12 hour period.

In some embodiments, the Cₘₐₓ of Compound A in the pharmacokinetic profile after administration to a mammal on an empty stomach is no more than 3000 ng/mL. In some embodiments, the Cₘₐₓ is no more than 4000 ng/mL. In some embodiments, the Cₘₐₓ is no more than 5000 ng/mL.

In some embodiments, the Cₘᵢₙ of Compound A in the pharmacokinetic profile after administration to a mammal on an empty stomach is no less than 400 ng/mL. In some embodiments, the Cₘᵢₙ is no less than 500 ng/mL. In some embodiments, the Cₘᵢₙ is no less than 600 ng/mL. In some embodiments, the Cₘᵢₙ is no less than 800 ng/mL.

In some embodiments, the plasma concentration of Compound A after administration to a mammal on an empty stomach is maintained, for example at least 600 ng/mL, for more than 8 hours. In some embodiments, the plasma concentration is maintained for more than 10 hours. In some embodiments, the plasma concentration is maintained for more than 12 hours. In some embodiments, the plasma concentration is maintained for more than 18 hours. In some embodiments, the plasma concentration is maintained for more than 24 hours.

In some embodiments, the pharmacokinetic profile after administration to a mammal on an empty stomach demonstrates a Cₘₐₓ of Compound A of no more than 3000 ng/mL, and a plasma concentration of Compound A of at least 600 ng/mL for more than 8 hours. In some embodiments, the Cₘₐₓ is no more than 3000 ng/mL and the plasma concentration is at least 600 ng/mL for more than 12 hours. In some embodiments, the Cₘₐₓ is no more than 3000 ng/mL and the plasma concentration is at least 600 ng/mL for more than 18 hours. In some embodiments, the Cₘₐₓ is no more than 3000 ng/mL and the plasma concentration is at least 600 ng/mL for more than 24 hours.

In some embodiments, the compositions disclosed herein are orally administered to a mammal, and the extended pharmaceutical formulation enables an 8 to at least 12 hour drug release.

In some embodiments, the extended pharmaceutical formulation enables an 8 to at least 18 hour drug release. In some embodiments, the extended pharmaceutical formulation enables an at least 8 hour drug release. In some embodiments, the extended pharmaceutical formulation enables an at least 10 hour drug release. In some embodiments, the extended pharmaceutical formulation enables an at least 12 hour drug release. In some embodiments, the extended pharmaceutical formulation enables an at least 18 hour drug release. In some embodiments, the extended pharmaceutical formulation enables an at least 24 hour drug release.

### Processes of Manufacture

In some embodiments, the tablet has a circular cross-section with a diameter of about ¼ to about ⅓ inch. In some embodiments, the tablet has a circular cross-section with a diameter of about ¼ inch. In some embodiments, the tablet has a circular cross-section with a diameter of about ⅓ inch.

In some embodiments, the tablet has a circular cross-section with a diameter of about 6.35 mm to about 8.46 mm. In some embodiments, the tablet has a circular cross-section with a diameter of about 6.35 mm. In some embodiments, the tablet has a circular cross-section with a diameter of about 8.46 mm.

In some embodiments, the oral form has content uniformity (e.g., for Compound A). In some embodiments, the content uniformity is as measured by a content uniformity test in the International Pharmacopoeia (IP), British Pharmacopoeia (BP), United States Pharmacopoeia (USP), or European Pharmacopoeia (Ph. Eur.), which are each incorporated herein by reference. In some embodiments, the oral form has a relative standard deviation that is less than, or is less than about, 10%, 9%, 8%, 7%, 6%, 5%, 4%, 3%, 2%, 1%, or 0.5% content. In some embodiments, the oral form has no value that falls outside a range, for example 75-125%, 80-125%, 85-120%, 85-115%, 90-120%, 90-110%, or 95-105% content. In some embodiments, the oral form has no less than, or less than about, 85%, 90%, 95%, 96%, 97%, 98%, 99%, or 99.5% content. In some embodiments, the oral form has no more than, or more than about, 100.5%, 101%, 102%, 103%, 104%, 105%, 110%, 115%, 120%, or 125% content.

In some embodiments, the tablet has a hardness of about 5 kp to about 9 kp. In some embodiments, the tablet has a hardness of about 6 kp to about 8 kp. In some embodiments, the tablet has a hardness of about 7 kp. In some embodiments, the tablet has a hardness of about 8 kp to about 12 kp. In some embodiments, the tablet has a hardness of about 12 kp to about 18 kp. In some embodiments, the tablet has a hardness of about 14 kp to about 16 kp. In some embodiments, the tablet has a hardness of about 15 kp.

In some embodiments, the tablet has a core weight of about 600 mg to about 980 mg. In some embodiments, the tablet has a core weight of about 600 mg to about 900 mg. In some embodiments, the tablet has a core weight of about 600 mg to about 630 mg. In some embodiments, the tablet has a core weight of about 615 mg. In some embodiments, the tablet has a core weight of about 690 mg to about 710 mg. In some embodiments, the tablet has a core weight of about 700 mg. In some embodiments, the tablet has a core weight of about 740 mg to about 760 mg. In some embodiments, the tablet has a core weight of about 750 mg. In some embodiments, the tablet has a core weight of about 800 mg to about 830 mg. In some embodiments, the tablet has a core weight of about 815 mg.

In some embodiments, the tablet has a total weight of about 600 mg to about 980 mg. In some embodiments, the tablet has a total weight of about 600 mg to about 900 mg. In some embodiments, the tablet has a total weight of about 600 mg to about 630 mg. In some embodiments, the tablet has a total weight of about 615 mg. In some embodiments, the tablet has a total weight of about 690 mg to about 710 mg. In some embodiments, the tablet has a total weight of about 700 mg. In some embodiments, the tablet has a total weight of about 740 mg to about 760 mg. In some embodiments, the tablet has a total weight of about 750 mg. In some embodiments, the tablet has a total weight of about 840 mg to about 860 mg. In some embodiments, the tablet has a total weight of about 850 mg. In some embodiments, the tablet has a total weight of about 790 mg to about 810 mg. In some embodiments, the tablet has a total weight of about 800 mg.

In some embodiments, the tablet has an amount of Compound A of about 200 mg to about 350 mg. In some embodiments, the tablet has an amount of Compound A of about 250 mg to about 300 mg. In some embodiments, the tablet has an amount of Compound A of about 200 mg. In some embodiments, the tablet has an amount of Compound A of about 250 mg. In some embodiments, the tablet has an amount of Compound A of about 300 mg.

In some embodiments, the tablet has an amount of Compound A of about 5% to about 70% by weight. In some embodiments, the tablet has an amount of Compound A of about 10% to about 50% by weight. In some embodiments, the tablet has an amount of Compound A of about 35% to about 45% by weight. In some embodiments, the tablet has an amount of Compound A of about 5%, about 10%, about 15%, about 20%, about 25%, about 30%, about 35%, about 40%, about 45%, about 50%, about 55%, about 60%, about 65%, or about 70% by weight. In some embodiments, the tablet has an amount of Compound A of about 40% by weight. In some embodiments, the tablet has an amount of Compound A of about 39% by weight. In some embodiments, the tablet has an amount of Compound A of about 39.6% by weight. In some embodiments, the tablet has an amount of Compound A of about 42.4% by weight. In some embodiments, the tablet has an amount of Compound A of about 35.7% by weight. In some embodiments, the tablet has an amount of Compound A of about 37.5% by weight.

In some embodiments provided herein is a pharmaceutical composition prepared by the process comprising: curing a mixture comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A) or pharmaceutically acceptable salts, solvates, and hydrates thereof, a first excipient, and a second excipient to form the composition.

In some embodiments, the first excipient is selected from hydrophilic release controlling polymer, such as hydroxypropyl methyl cellulose or HPMC (such as Methocel™, various molecular weight), hydroxypropylcellulose or HPC (such as Klucel™ with various molecular weight), Poly(ethylene) Oxide (such as PolyOx™), soluble polyvinylpyrrolidone or Povidon (such as Kollidone® of various grades), cross-linked polyacrylic acid polymer (Carbopol®), or others.

In some embodiments, the first excipient is selected from hydroxypropylcellulose or HPC (such as Klucel™ HXF or EXF), cross-linked polyacrylic acid polymer (such as Carbopol® 71G NF), or hydroxypropyl methyl cellulose or HPMC (such as Methocel™ K100M).

In other embodiments, the first excipient is selected from hydrophobic release controlling polymers, such as ethylcellulose (such as, Ethocel™), hypromellose acetate succinate, cellulose acetate, cellulose acetate propionate, Eudogrit®, and natural wax, or others.

In some embodiments, the first excipient is selected from Eudogrit® (such as Eudogrit® RLPO), or ethylcellulose (such as, Ethocel™ 10 cp).

In other embodiments, the first excipient is selected from hydrophilic release controlling polymer, such as hydroxypropyl methyl cellulose or HPMC (such as Methocel™, various molecular weight), hydroxypropylcellulose or HPC (such as Klucel™ with various molecular weight), Poly(ethylene) Oxide (such as PolyOx™), soluble polyvinylpyrrolidone or Povidon (such as Kollidone® of various grades), cross-linked polyacrylic acid polymer (Carbopol®), or others; and the second excipient is selected from hydrophobic release controlling polymers, such as ethylcellulose (such as, Ethocel™), hypromellose acetate succinate, cellulose acetate, cellulose acetate propionate, Eudogrit®, and natural wax, or others.

In some embodiments, the release rate is the release rate measured with USP Apparatus 1 (baskets) at 100 rpm in 500 mL of an aqueous medium at a pH of 6.8 at a temperature of 37° C.±0.5° C. In some embodiments, the release rate is the release rate measured with USP Apparatus 1 (baskets) at 100 rpm in 900 mL of an aqueous medium at a pH of 6.8 at a temperature of 37° C.±0.5° C.

In some embodiments, the release rate is the release rate measured with USP Apparatus 1 (baskets) at 100 rpm in 500 mL of an aqueous medium at a temperature of 37° C.±0.5° C., comprising sodium phosphate at a concentration of 0.05 M. In some embodiments, the release rate is the release rate measured with USP Apparatus 1 (baskets) at 100 rpm in 900 mL of an aqueous medium at a temperature of 37° C.±0.5° C., comprising sodium phosphate at a concentration of 0.05 M.

USP Apparatus 1 (BASKETS) is described, for example, in the United States Pharmacopeia Convention, Feb. 1, 2012, Chapter <711> (“Dissolution”), incorporated by reference herein in its entirety. See http://www.usp.org/sites/default/files/usp_pdf/EN/USPNF/revisions/m99470-gc_711.pdf. The USP Apparatus 1 (baskets) assembly consists of the following: a vessel, which may be covered, made of glass or other inert, transparent material; a motor; a metallic drive shaft; and a cylindrical basket. The materials should not sorb, react, or interfere with the specimen being tested. The vessel is partially immersed in a suitable water bath of any convenient size or heated by a suitable device such as a heating jacket. The water bath or heating device permits holding the temperature inside the vessel at 37±0.5° during the test and keeping the bath fluid in constant, smooth motion. No part of the assembly, including the environment in which the assembly is placed, contributes significant motion, agitation, or vibration beyond that due to the smoothly rotating stirring element. An apparatus that permits observation of the specimen and stirring element during the test is preferable. The vessel is cylindrical, with a hemispherical bottom and with one of the following dimensions and capacities: for a nominal capacity of 1 L, the height is 160 mm to 210 mm and its inside diameter is 98 mm to 106 mm; for a nominal capacity of 2 L, the height is 280 mm to 300 mm and its inside diameter is 98 mm to 106 mm; and for a nominal capacity of 4 L, the height is 280 mm to 300 mm and its inside diameter is 145 mm to 155 mm. Its sides are flanged at the top. A fitted cover may be used to retard evaporation. If a cover is used, it provides sufficient openings to allow ready insertion of the thermometer and withdrawal of specimens. The shaft is positioned so that its axis is not more than 2 mm at any point from the vertical axis of the vessel and rotates smoothly and without significant wobble that could affect the results. The vertical center line of the blade passes through the axis of the shaft so that the bottom of the blade is flush with the bottom of the shaft. A distance of 25±2 mm between the bottom of the blade and the inside bottom of the vessel is maintained during the test. The metallic or suitably inert, rigid blade and shaft comprise a single entity. A suitable two-part detachable design may be used provided the assembly remains firmly engaged during the test.

A speed-regulating device is used that allows the shaft rotation speed to be selected and maintained at the specified rate within +4%. Shaft and basket components of the stirring element are fabricated of stainless steel, type 316, or other inert material. A basket having a gold coating of about 0.0001 inch (2.5 μm) thick may be used. A dosage unit is placed in a dry basket at the beginning of each test. The distance between the inside bottom of the vessel and the bottom of the basket is maintained at 25±2 mm during the test.

USP Apparatus 2 (Paddle Apparatus) is described, for example, in the United States Pharmacopeia Convention, Feb. 1, 2012, Chapter <711> (“Dissolution”), incorporated by reference herein in its entirety. See http://www.usp.org/sites/default/files/usp_pdf/EN/USPNF/revisions/m99470-gc_711.pdf. The assembly from Apparatus 1 is used, except that a paddle formed from a blade and a shaft is used as the stirring element. The shaft is positioned so that its axis is not more than 2 mm from the vertical axis of the vessel at any point and rotates smoothly without significant wobble that could affect the results. The vertical center line of the blade passes through the axis of the shaft so that the bottom of the blade is flush with the bottom of the shaft. A distance of 25±2 mm between the bottom of the blade and the inside bottom of the vessel is maintained during the test. The metallic or suitably inert, rigid blade and shaft comprise a single entity. A suitable two-part detachable design may be used provided the assembly remains firmly engaged during the test. The paddle blade and shaft may be coated with a suitable coating so as to make them inert. The dosage unit is allowed to sink to the bottom of the vessel before rotation of the blade is started. A small, loose piece of nonreactive material, such as not more than a few turns of wire helix, may be attached to dosage units that would otherwise float. Other validated sinker devices may be used.

Where water or a specified rate medium with a pH of less than 6.8 is specified as the medium, the same medium may be used with the addition of purified pepsin that results in an activity of 750,000 Units or less per 1000 mL. For media with a pH of 6.8 or greater, pancreatin can be added to produce not more than 1750 USP Units of protease activity per 1000 mL.

A compound or composition provided herein can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.).

Certain compounds described herein can be asymmetric (e.g, having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Resolution of racemic mixtures of compounds can be carried out by any of numerous methods known in the art. An example method includes fractional recrystallization (for example, diastereomeric salt resolution) using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of 3-methylbenzylamine (e.g, S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g, dinitrobenzoylphenylglycine). Suitable elution solvent composition can be determined by one skilled in the art.

Compounds described herein can also include all isotopes of atoms occurring in the intermediates or final compounds. Isotopes include those atoms having the same atomic number but different mass numbers. For example, isotopes of hydrogen include tritium and deuterium.

Compounds described herein can also include tautomeric forms, such as keto-enol tautomers. Tautomeric forms can be in equilibrium or sterically locked into one form by appropriate substitution.

It is appreciated that certain features disclosed herein, which are, for clarity, described in the context of separate embodiments, may also be provided in combination in a single embodiment. Conversely, various features which are, for brevity, described in the context of a single embodiment, may also be provided separately or in any suitable sub-combination.

### Indications and Methods of Treatment

The compositions disclosed herein are useful in the treatment of diseases and disorders related to modulation of protein kinase activity (for example PKCβ activity), and in the amelioration of symptoms thereof. Accordingly, some embodiments of the present invention relate to a method of modulating the activity of PKCβ by contacting the protein kinase with a composition according to any of the embodiments described herein.

In some embodiments provided herein is a method for the treatment of a protein kinase (for example PKCβ) mediated disorder in an individual, comprising administering to the individual in need thereof, a composition according to any of the embodiments described herein.

In some embodiments provided herein is a composition according to any of the embodiments described herein, for use in a method of treatment of the human or animal body by therapy.

In some embodiments provided herein is a composition according to any of the embodiments described herein, for use in a method of modulating the activity of a protein kinase (for example PKCβ.

In some embodiments provided herein is a composition according to any of the embodiments described herein, for use in a method of inhibiting PKCβ.

In some embodiments provided herein is a composition according to any of the embodiments described herein, for use in a method of treatment of a PKCβ mediated disorder.

The compositions disclosed herein are useful in the treatment of other diseases and disorders related to modulation of protein kinase (for example, PKCβ) activity, and in the amelioration of symptoms thereof, without limitation, these include the following:

1. Cancer

### Hematological Malignancies

Hematological malignancies are cancers that affect the blood and lymph system. The cancer may begin in blood-forming tissue (e.g., bone marrow), or in the cells of the immune system. In some embodiments, a hematologic malignancy is a leukemia, a non-Hodgkin lymphoma (NHL), a Hodgkin lymphoma, or a multiple myeloma. Hematological malignancies can originate either in the lymphatic tissues (e.g., lymphoma) or in the bone marrow (e.g., leukemia and myeloma), and all involve the uncontrolled growth of lymphocytes or white blood cells.

Malignant lymphomas are neoplastic transformations of cells that reside predominantly within lymphoid tissues. Two groups of malignant lymphomas are Hodgkin's lymphoma and non-Hodgkin's lymphoma (NHL). Both types of lymphomas infiltrate reticuloendothelial tissues. However, they differ in the neoplastic cell of origin, site of disease, presence of systemic symptoms, and response to treatment. Non-Hodgkin lymphomas (NHL) are a diverse group of malignancies that are predominately of B-cell origin. NHL may develop in any organs associated with the lymphatic system such as the spleen, lymph nodes, or tonsils and can occur at any age. NHL is often marked by enlarged lymph nodes, fever, and weight loss. NHL is classified as either B-cell or T-cell NHL. Although chemotherapy can induce remissions in the majority of indolent lymphomas, cures are rare and most patients eventually relapse, requiring further therapy.

A non-limiting list of the B-cell NHL includes Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma. Additional non-Hodgkin's lymphomas are contemplated within the scope of the present invention and apparent to those of ordinary skill in the art.

Some patients achieve a remission (an absence of signs and symptoms) after initial treatment for a hematological malignancy. However, other patients have residual cancerous cells that remain even after intensive treatment.

In some embodiments, an individual has a hematological malignancy that has relapsed after therapeutic treatment. In some embodiments, the hematological malignancy is resistant to therapeutic treatment. In some embodiments, the hematological malignancy has primary resistance to therapeutic treatment. In some embodiments, the hematological malignancy has secondary or acquired resistance to therapeutic treatment. In some embodiments, the hematological malignancy has primary resistance to treatment with a BTK inhibitor. In some embodiments, the hematological malignancy has primary resistance to treatment with ibrutinib. In some embodiments, the hematological malignancy has acquired resistance to treatment with a BTK inhibitor. In some embodiments, the hematological malignancy has acquired resistance to treatment with ibrutinib. In some embodiments, treatment of a hematological malignancy with a BTK inhibitor is unsuitable or otherwise contraindicated. In some embodiments, treatment of a hematological malignancy with ibrutinib is unsuitable or otherwise contraindicated.

Disclosed herein, in some embodiments, are methods of treating a hematological malignancy in an individual in need thereof, comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt thereof. Disclosed herein, in some embodiments, are methods of treating a hematological malignancy in an individual in need thereof, comprising administering to the individual a modified release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt thereof. Disclosed herein, in some embodiments, are methods of treating a hematological malignancy in an individual in need thereof, comprising administering to the individual a non-immediate release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt thereof. In some embodiments, the method further comprises administration of a BTK inhibitor. In some embodiments, the method further comprises administration of ibrutinib.

### DLBCL

Diffuse large B-cell lymphoma (DLBCL) is the most common aggressive lymphoma subtype in western countries, accounting for approximately 30% of new cases of non-Hodgkin's lymphoma (NHL). Genetic tests have shown that there are different subtypes of DLBCL. These subtypes seem to have different outlooks (prognoses) and responses to treatment. At least 3 molecular subtypes of DLBCL can be distinguished: germinal center B-cell-like (GCB) DLBCL, activated B-cell-like (ABC) DLBCL, and primary mediastinal B-cell lymphoma (PMBL). DLBCL can affect any age group, but occurs mostly in older people (the average age is mid-60s).

The ABC subtype of DLBCL (ABC-DLBCL) accounts for approximately 30% total DLBCL diagnoses. It is considered the least curable of the DLBCL molecular subtypes and, as such, patients diagnosed with the ABC-DLBCL typically display significantly reduced survival rates compared with individuals with other types of DLCBL. ABC-DLBCL is most commonly associated with chromosomal translocations deregulating the germinal center master regulator BCL6 and with mutations inactivating the PRDM1 gene, which encodes a transcriptional repressor required for plasma cell differentiation.

A particularly relevant signaling pathway in the pathogenesis of ABC-DLBCL is the one mediated by the nuclear factor (NF)-κB transcription complex. The NF-κB family comprises 5 members (p50, p52, p65, c-rel and RelB) that form homo- and heterodimers and function as transcriptional factors to mediate a variety of proliferation, apoptosis, inflammatory and immune responses and are critical for normal B-cell development and survival. NF-κB is widely used by eukaryotic cells as a regulator of genes that control cell proliferation and cell survival. As such, many different types of human tumors have misregulated NF-κB: that is, NF-κB is constitutively active. Active NF-κB turns on the expression of genes that keep the cell proliferating and protect the cell from conditions that would otherwise cause it to die via apoptosis.

The dependence of ABC DLBCLs on NF-kB depends on a signaling pathway upstream of IkB kinase comprised of CARD11, BCL10 and MALT1 (the CBM complex). Interference with the CBM pathway extinguishes NF-kB signaling in ABC DLBCL cells and induces apoptosis. The molecular basis for constitutive activity of the NF-kB pathway is a subject of current investigation but some somatic alterations to the genome of ABC DLBCLs clearly invoke this pathway. For example, somatic mutations of the coiled-coil domain of CARD11 in DLBCL render this signaling scaffold protein able to spontaneously nucleate protein-protein interaction with MALT1 and BCL10, causing IKK activity and NF-kB activation. Constitutive activity of the B cell receptor signaling pathway has been implicated in the activation of NF-kB in ABC DLBCLs with wild type CARD11, and this is associated with mutations within the cytoplasmic tails of the B cell receptor subunits CD79A and CD79B. Oncogenic activating mutations in the signaling adapter MYD88 activate NF-kB and synergize with B cell receptor signaling in sustaining the survival of ABC DLBCL cells. In addition, inactivating mutations in a negative regulator of the NF-kB pathway, A20, occur almost exclusively in ABC DLBCL.

Indeed, genetic alterations affecting multiple components of the NF-κB signaling pathway have been recently identified in more than 50% of ABC-DLBCL patients, where these lesions promote constitutive NF-κB activation, thereby contributing to lymphoma growth. These include mutations of CARD 11 (˜10% of the cases), a lymphocyte-specific cytoplasmic scaffolding protein that—together with MALT1 and BCL10—forms the BCR signalosome, which relays signals from antigen receptors to the downstream mediators of NF-κB activation. An even larger fraction of cases (˜30%) carry biallelic genetic lesions inactivating the negative NF-κB regulator A20. Further, high levels of expression of NF-κB target genes have been observed in ABC-DLBCL tumor samples.

Disclosed herein, in some embodiments, are methods of treating a DLBCL in an individual in need thereof, comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt thereof. In some embodiments, the DLBCL is ABC-DLBCL. In some embodiments, the method further comprises administration of a BTK inhibitor. In some embodiments, the method further comprises administration of ibrutinib. In some embodiments, the method further comprises administration of ibrutinib, lenalidomide and a CD20 antibody. In some embodiments, the method further comprises administration of lenalidomide and a CD20 antibody.

### Follicular Lymphoma

As used herein, the term “follicular lymphoma” refers to any of several types of non-Hodgkin's lymphoma in which the lymphomatous cells are clustered into nodules or follicles. The term follicular is used because the cells tend to grow in a circular, or nodular, pattern in lymph nodes. The average age for people with this lymphoma is about 60. Follicular lymphoma, a B-cell lymphoma, is the most common indolent (slow-growing) form of NHL, accounting for approximately 20 percent to 30 percent of all NHLs.

### CLL/SLL

Chronic lymphocytic leukemia and small lymphocytic lymphoma (CLL/SLL) are commonly thought as the same disease with slightly different manifestations. Where the cancerous cells gather determines whether it is called CLL or SLL. When the cancer cells are primarily found in the lymph nodes, it is called SLL. SLL accounts for about 5% to 10% of all lymphomas. When most of the cancer cells are in the bloodstream and the bone marrow, it is called CLL.

Both CLL and SLL are slow-growing diseases, although CLL, which is much more common, tends to grow slower. CLL and SLL are treated the same way. They are usually not considered curable with standard treatments, but depending on the stage and growth rate of the disease, most patients live longer than 10 years. Occasionally over time, these slow-growing lymphomas may transform into a more aggressive type of lymphoma.

Chronic lymphoid leukemia (CLL) is the most common type of leukemia. CLL is a lymphoid malignancy of clonal B cells that typically exhibit aberrant activation of the B-cell receptor (BCR) signaling pathway.

Small lymphocytic leukemia (SLL) is very similar to CLL described supra, and is also a cancer of B-cells. In SLL the abnormal lymphocytes mainly affect the lymph nodes. However, in CLL the abnormal cells mainly affect the blood and the bone marrow. The spleen may be affected in both conditions. SLL accounts for about 1 in 25 of all cases of non-Hodgkin lymphoma. It can occur at any time from young adulthood to old age, but is rare under the age of 50. SLL is considered an indolent lymphoma. This means that the disease progresses very slowly, and patients tend to live many years after diagnosis. However, most patients are diagnosed with advanced disease, and although SLL responds well to a variety of chemotherapy drugs, it is generally considered to be incurable. Although some cancers tend to occur more often in one gender or the other, cases and deaths due to SLL are evenly split between men and women. The average age at the time of diagnosis is 60 years.

Although SLL is indolent, it is persistently progressive. The usual pattern of this disease is one of high response rates to radiation therapy and/or chemotherapy, with a period of disease remission. This is followed months or years later by an inevitable relapse. Re-treatment leads to a response again, but again the disease will relapse. This means that although the short-term prognosis of SLL is quite good, over time, many patients develop fatal complications of recurrent disease. Considering the age of the individuals typically diagnosed with CLL and SLL, there is a need in the art for a simple and effective treatment of the disease with minimum side-effects that do not impede on the patient's quality of life. The instant invention fulfills this long standing need in the art.

### Mantle Cell Lymphoma

As used herein, the term, “Mantle cell lymphoma” refers to a subtype of B-cell lymphoma, due to CD5 positive antigen-naive pregerminal center B-cell within the mantle zone that surrounds normal germinal center follicles. MCL cells generally over-express cyclin D1 due to a t(11:14) chromosomal translocation in the DNA. Men are affected most often. The average age of patients is in the early 60s. The lymphoma is usually widespread when it is diagnosed, involving lymph nodes, bone marrow, and, very often, the spleen. Mantle cell lymphoma is not a very fast growing lymphoma, but is difficult to treat.

### Marginal Zone B-Cell Lymphoma

As used herein, the term “marginal zone B-cell lymphoma” refers to a group of related B-cell neoplasms that involve the lymphoid tissues in the marginal zone, the patchy area outside the follicular mantle zone. Marginal zone lymphomas account for about 5% to 10% of lymphomas. The cells in these lymphomas look small under the microscope. There are 3 main types of marginal zone lymphomas including extranodal marginal zone B-cell lymphomas, nodal marginal zone B-cell lymphoma, and splenic marginal zone lymphoma.

### MALT

The term “mucosa-associated lymphoid tissue (MALT) lymphoma”, as used herein, refers to extranodal manifestations of marginal-zone lymphomas. Most MALT lymphoma are a low grade, although a minority either manifest initially as intermediate-grade non-Hodgkin lymphoma (NHL) or evolve from the low-grade form. Most of the MALT lymphoma occur in the stomach, and roughly 70% of gastric MALT lymphoma are associated with Helicobacter pylori infection. Several cytogenetic abnormalities have been identified, the most common being trisomy 3 or t(11;18). Many of these other MALT lymphoma have also been linked to infections with bacteria or viruses. The average age of patients with MALT lymphoma is about 60.

### Nodal Marginal Zone B-Cell Lymphoma

The term “nodal marginal zone B-cell lymphoma” refers to an indolent B-cell lymphoma that is found mostly in the lymph nodes. The disease is rare and only accounts for 1% of all Non-Hodgkin's Lymphomas (NHL). It is most commonly diagnosed in older patients, with women more susceptible than men. The disease is classified as a marginal zone lymphoma because the mutation occurs in the marginal zone of the B-cells. Due to its confinement in the lymph nodes, this disease is also classified as nodal.

### Splenic Marginal Zone B-Cell Lymphoma

The term “splenic marginal zone B-cell lymphoma” refers to specific low-grade small B-cell lymphoma that is incorporated in the World Health Organization classification. Characteristic features are splenomegaly, moderate lymphocytosis with villous morphology, intrasinusodial pattern of involvement of various organs, especially bone marrow, and relative indolent course. Tumor progression with increase of blastic forms and aggressive behavior are observed in a minority of patients. Molecular and cytogenetic studies have shown heterogeneous results probably because of the lack of standardized diagnostic criteria.

### Burkitt Lymphoma

The term “Burkitt lymphoma” refers to a type of Non-Hodgkin Lymphoma (NHL) that commonly affects children. It is a highly aggressive type of B-cell lymphoma that often starts and involves body parts other than lymph nodes. In spite of its fast-growing nature, Burkitt's lymphoma is often curable with modern intensive therapies. There are two broad types of Burkitt's lymphoma—the sporadic and the endemic varieties:

Endemic Burkitt's lymphoma: The disease involves children much more than adults, and is related to Epstein Barr Virus (EBV) infection in 95% cases. It occurs primarily is equatorial Africa, where about half of all childhood cancers are Burkitt's lymphoma. It characteristically has a high chance of involving the jawbone, a rather distinctive feature that is rare in sporadic Burkitt's. It also commonly involves the abdomen.

Sporadic Burkitt's lymphoma: The type of Burkitt's lymphoma that affects the rest of the world, including Europe and the Americas is the sporadic type. Here too, it's mainly a disease in children. The link between Epstein Barr Virus (EBV) is not as strong as with the endemic variety, though direct evidence of EBV infection is present in one out of five patients. More than the involvement of lymph nodes, it is the abdomen that is notably affected in more than 90% of the children. Bone marrow involvement is more common than in the sporadic variety.

### Waldenstrom Macroglobulinemia

The term “Waldenstrom macroglobulinemia”, also known as lymphoplasmacytic lymphoma, is cancer involving a subtype of white blood cells called lymphocytes. It is characterized by an uncontrolled clonal proliferation of terminally differentiated B lymphocytes. It is also characterized by the lymphoma cells making an antibody called immunoglobulin M (IgM). The IgM antibodies circulate in the blood in large amounts, and cause the liquid part of the blood to thicken, like syrup. This can lead to decreased blood flow to many organs, which can cause problems with vision (because of poor circulation in blood vessels in the back of the eyes) and neurological problems (such as headache, dizziness, and confusion) caused by poor blood flow within the brain. Other symptoms can include feeling tired and weak, and a tendency to bleed easily. The underlying etiology is not fully understood but a number of risk factors have been identified, including the locus 6p21.3 on chromosome 6. There is a 2- to 3-fold risk increase of developing WM in people with a personal history of autoimmune diseases with autoantibodies and particularly elevated risks associated with hepatitis, human immunodeficiency virus, and rickettsiosis.

### Multiple Myeloma

Multiple myeloma is a cancer of the white blood cells known as plasma cells. A type of B cell, plasma cells are a crucial part of the immune system responsible for the production of antibodies in humans and other vertebrates. They are produced in the bone marrow and are transported through the lymphatic system. When plasma cells become cancerous and grow out of control, they can produce a tumor called a plasmacytoma. These tumors generally develop in a bone, but they are also rarely found in other tissues. When a plasmacytoma starts in other tissues (such as the lungs or other organs), it is called an extramedullary plasmacytoma. An individual with only a single plasma cell tumor, has an isolated (or solitary) plasmacytoma. An individual with more than one plasmacytoma, has multiple myeloma.

### Leukemia

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive buildup of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

Within these main categories, there are several subcategories including, but not limited to, acute lymphoblastic leukemia (ALL), acute myelogenous leukemia (AML), chronic myelogenous leukemia (CML), and chronic lymphoblastic leukemia (CLL).

### AML

Acute myeloid leukemia (AML), also known as acute myelogenous leukemia, acute myeloblastic leukemia, acute granulocytic leukemia or acute nonlymphocytic leukemia, is a fast-growing form of cancer of the blood and bone marrow. Although overall AML is a relatively rare disease, it is the most common acute leukemia affecting adults. AML occurs when the bone marrow begins to make blasts, cells that have not yet completely matured. These blasts normally develop into white blood cells. However, in AML, these cells do not develop and are unable to ward off infections. In AML, the bone marrow may also make abnormal red blood cells and platelets. The number of these abnormal cells increases rapidly, and the abnormal (leukemia) cells begin to crowd out the normal white blood cells, red blood cells and platelets that the body needs.

One of the main factors that differentiate AML from the other main forms of leukemia is that it has eight different subtypes, which are based on the cell that the leukemia developed from. The types of acute myelogenous leukemia include: Myeloblastic (M0)—on special analysis; Myeloblastic (M1)—without maturation; Myeloblastic (M2)—with maturation; Promyelocytic (M3); Myelomonocytic (M4); Monocytic (M5); Erythroleukemia (M6); and Megakaryocytic (M7). In vitro studies have shown that bone marrow mesenchymal stromal cells (BM-MSC) protect AML blasts from spontaneous and chemotherapy-induced apoptosis (A. M. Abdul-Azizm et al Cancer Res (2017) 77(2): 303-311). Abdul-Azizm et al report that macrophage inhibitory factor (MIF)-induced stromal PKCβ/IL8 is the essential feature of this stromal support in human AML. The authors demonstrate that pharmacologic inhibition of PKCβ inhibits MIF-induced IL8 induction in BM-MSCs. These results show that a bidirectional, prosurvival mechanism between AML blasts and BM-MSCs exists and that this mechanism is blocked by inhibition of PKCβ.

Bcl2 is a cellular oncogene product associated with the t(14,18) translocation commonly seen in B-cell lymphomas. However, Bcl2 expression levels alone do not always correlate with poor prognosis in patients diagnosed with AML. The phosphorylation status of Bcl2 can influence Bcl2 activity. PKCα and extracellular signal-related kinase (ERK) have been identified as Bcl2 kinases that promote survival. It has also been demonstrated that Bcl2 is phosphorylated in nearly half the patient AML blast cells tested. Furthermore, Bcl2 was always phosphorylated in AML blast cells with activated PKCα and ERK but never in cells that lack both activated kinases. AML patients with blast cells expressing phosphorylated Bcl2 exhibit shorter overall survival (particularly when PKCα was active) compared to patients with blast cells expressing unphosphorylated Bcl2. Survival of AML patients with active PKCα was shorter compared to patients with no phosphorylated PKC and appeared to be shortest in patients in which PKCα and BCL2 were phosphorylated. Patients with upregulated activation of BCL2 and PKCα typically demonstrate the poorest clinical outcomes. It has been shown that the PKC inhibitor enzastaurin promotes the apoptosis of AML derived cell lines and in blast cells derived from patients with newly diagnosed or recurrent AML. This effect was not due to inhibition of PKCβ, but rather was correlated with PKCα inhibition.

Described herein, in some embodiments, are methods of treating an AML in a subject in need thereof comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt thereof. In some embodiments, the method further comprises administration of a BLC2 inhibitor.

It has been demonstrated that PKCβ inhibition may play an important role in myeloid malignancies as well as PKCα. Li, et al (Leukemia & Lymphoma (2011), 52(7):1312-1320) shows that PKCβ signaling is upregulated in the human CML cell line K562 and that inhibition of PKCβ inhibited K562 cell proliferation in a time- and dose-dependent manner. Because the PKCβ inhibitor (a novel bisindolymaleimide derivative WK234) retarded cell proliferation and induced apoptosis through suppression of the PKCβ signal pathway, inhibition of PKCβ might be a promising approach for the treatment of CML. Further, Dufies, et al (Oncotarget 2011; 2: 874-885) provides supporting evidence that AXL upregulation is responsible for resistance of CML cells to imatinib and is a hallmark of imatinib resistance. The authors demonstrate that this upregulation of AXL requires both PKCα and PKCβ. Thus, inhibition of both PKCα and PKCβ could be a possible mechanism for treatment of patients with imatinib resistant CML.

In research related to acute lymphoblastic leukemia (ALL), Saba, et al (Leukemia & Lymphoma, 2011; 52(5): 877-886) found that PKCβ inhibitor treatment resulted in a dose-dependent reduction in viability in all five ALL cell lines tested.

Described herein, in some embodiments, is a method of treating leukemia in a subject in need thereof comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt thereof, wherein the leukemia is chosen from acute lymphoblastic leukemia (ALL), acute myelogenous leukemia (AML), chronic myelogenous leukemia (CML), or chronic lymphoblastic leukemia (CLL).

### T-Cell Lymphomas

T-cell lymphomas make up less than 15% of non-Hodgkin lymphomas in the United States. There are many types of T-cell lymphoma, but they are all fairly rare.

### Precursor T-Lymphoblastic Lymphoma/Leukemia

Precursor T-Lymphoblastic Lymphoma/Leukemia accounts for about 1% of all lymphomas. It can be considered either a lymphoma or leukemia, depending on how much of the bone marrow is involved (leukemias have more bone marrow involvement). The cancer cells are small-to-medium sized, immature T-cells.

Precursor T-lymphoblastic lymphoma often starts in the thymus, where many T cells are made. Patients are most often young adults, with males being affected more often than females. Precursor T-lymphoblastic lymphoma is fast-growing, but the prognosis following chemotherapy treatment is good if the cancer has not spread to the bone marrow. The lymphoma form of this disease is often treated in the same way as the leukemia form.

### Peripheral T-Cell Lymphomas

Peripheral T-cell lymphomas (PTCLs) are uncommon and aggressive types of non-Hodgkin lymphoma (NHL) that develop in mature white blood cells. PTCLs generally affect people aged 60 years and older and are diagnosed slightly more often in men than in women.

Cutaneous T-cell lymphomas (mycosis fungoides, Sezary syndrome, and others) start in the skin. Skin lymphomas account for about 5% of all lymphomas.

Adult T-cell lymphoblastic leukemia/lymphoma is typically caused by infection with a virus called HTLV-1. This disease is rare in the United States and much more common in Japan, the Caribbean, and parts of Africa—where the HTLV-1 virus is more common. There are 4 subtypes: smoldering, chronic, acute, and lymphoma.

The smoldering subtype has abnormal T-cells in the blood without an increased number of lymphocytes in the blood. This lymphoma may involve the skin or lungs, but there is no involvement of other tissues. The smoldering type grows slowly and has a good prognosis.

The chronic subtype also grows slowly and has a good prognosis. It has an increase in total lymphocytes and T-cells in the blood. It may involve the skin, lungs, lymph nodes, liver, and/or spleen, but nor other tissues.

The acute subtype acts like acute leukemia. It has high lymphocyte and T-cell counts, often along with enlargement of lymph nodes, liver, and spleen. The skin and other organs may be involved with lymphoma as well. Patients often have fever, night sweats, and/or weight loss, as well as certain abnormal blood test results.

The lymphoma subtype grows more quickly than the chronic and smoldering types, but not as fast as the acute type. It has enlarged lymph nodes without increased lymphocytes in the blood, and the T-cell count is not high.

Angioimmunoblastic T-cell lymphoma (AITL accounts for between 1-2 percent of all cases of NHL and typically follows an aggressive course. AITL is more common in older adults. AITL tends to involve the lymph nodes as well as the spleen or liver, which can cause them to be enlarged. Patients usually have fever, weight loss, and skin rashes and often develop infections. This lymphoma often progresses quickly. Treatment is often effective at first, but the lymphoma tends to relapse.

Extranodal, nasal natural killer/T-cell lymphoma is a rare lymphoma that often involves the upper airway passages, such as the nose and upper throat, but it can also invade the skin and digestive tract. Cells of this lymphoma are similar in some ways to normal natural killer (NK) cells. NK cells are lymphocytes that can respond to infections more quickly than T-cells and B-cells. Extranodal, nasal NK/T-cell lymphoma is more commonly found in Asia and Latin America and is associated with the Epstein-Barr virus (EBV).

Enteropathy-associated intestinal T-cell lymphoma (EATL): EATL is a lymphoma that occurs in the lining of the intestine. This lymphoma is most common in the jejunum (the second part of the small intestine), but can also occur elsewhere in the small intestine and in the colon. EATL often affects more than one place in the intestine, and may spread to the nearby lymph nodes, as well. It can cause the intestine to become obstructed or perforated. There are two subtypes of this lymphoma.

Type I EATL occurs in people with a disease called gluten-sensitive enteropathy (also known as celiac disease, celiac sprue, or sprue). Sprue is an autoimmune disease in which gluten, the main protein in wheat flour, causes the body produce antibodies that attack the lining of the intestine and other parts of the body. This lymphoma is more common in men than women, and tends to occur in people in their 60s and 70s. People who do not tolerate gluten, but don't have sprue, do not seem to have an increased risk of this type of lymphoma. Type II EATL is not linked to sprue and is less common than type I.

Anaplastic large cell lymphoma (ALCL) is a rare T-cell lymphoma that constitutes about 3 percent of all cases of lymphomas in adults. ALCL is much more prevalent in children. ALCL usually starts in lymph nodes and can also spread to skin. This type of lymphoma tends to be fast-growing, but many people with this lymphoma are cured with aggressive chemotherapy.

The two main forms of ALCL are primary cutaneous, which only affects the skin, and systemic. Systemic ALCL is divided into subtypes based upon the presence or absence of anaplastic lymphoma kinase (ALK). ALK-positive ALCL tends to occur in younger patients and tends to have a better prognosis than the ALK-negative type.

Peripheral T-cell lymphoma, not otherwise unspecified is the most common type of PTCL and is the name given to T-cell lymphomas that don't readily fit into any of the groups above. They make up about half of all T-cell lymphomas. Most people diagnosed with this disease are in their 60s. This lymphoma often has nodal involvement, but extranodal sites, such as the liver, bone marrow, gastrointestinal tract and skin, may also be involved. As a group, these lymphomas tend to be widespread and grow quickly. Some patients respond well to chemotherapy, but long-term survival is not common.

### Ewing's Sarcoma

Ewing's sarcoma is a cancerous tumor that grows in the bones or in the tissue around bones (soft tissue), typically the legs, pelvis, ribs, arms or spine. Ewing sarcoma can spread to the lungs, bones and bone marrow. Ewing sarcoma is the second most frequent childhood bone tumor, but it is very rare. Ewing sarcoma is a highly metastatic tumor with around 25% of patients presenting metastasis at the time of diagnosis. About half of all Ewing sarcoma tumors occur in children and young adults between ages 10 and 20. Although not often seen, Ewing sarcoma can occur as a second cancer, especially in patients treated with radiation therapy.

The most common translocation in Ewing's Sarcoma, present in about 90% of cases, generates an aberrant transcription factor through fusion of the EWSR1 gene with the FLI1 gene. PKCβ has been found to be a target modulated by EWSR1-FLI1 in primary Ewing tumors compared with other tumors types. PKCβ has been demonstrated to be crucial for Ewing's Sarcoma tumor cell survival in vitro and tumor development in vivo.

Described herein, in some embodiments, are methods of treating a Ewing's Sarcoma in a subject in need thereof comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine (Compound A), or a pharmaceutically acceptable salt thereof.

2. Autoimmune Disorders

### Lupus

Lupus is a chronic inflammatory disease that occurs when the immune system attacks host tissues and organs. Inflammation caused by lupus can affect many different body systems, including joints, skin, kidneys, blood cells, brain, heart and lungs. Lupus can be difficult to diagnose because its signs and symptoms often mimic those of other ailments. The most distinctive sign of lupus is a facial rash that resembles the wings of a butterfly unfolding across both cheeks and occurs in many but not all cases of lupus. Some individuals are born with a tendency toward developing lupus, which may be triggered by infections, certain drugs or even sunlight. Currently available treatment can help control symptoms. Most individuals with lupus have mild disease characterized by episodes called flares, during which signs and symptoms are increased, then diminish or even disappear completely for a time. The signs and symptoms of lupus depend on which body systems are affected by the disease. The most common signs and symptoms include, fatigue and fever, joint pain, stiffness and swelling, butterfly-shaped rash on the face that covers the cheeks and bridge of the nose, skin lesions that appear or worsen with sun exposure, fingers and toes that turn white or blue when exposed to cold or during stressful periods (Raynaud's phenomenon), shortness of breath, chest pain, dry eyes, headaches, confusion and memory loss.

The origin lupus is suspected to result from a combination of genetics and environment causes. It appears that individuals with an inherited predisposition for lupus may develop the disease when they come into contact with environmental factors that can trigger lupus. Some potential triggers include sunlight, as exposure to the sun may bring on lupus skin lesions or trigger an internal response in susceptible individuals, and episodes of infection, as having an infection can initiate lupus or cause a relapse. Lupus can be triggered by certain types of anti-seizure medications, blood pressure medications and antibiotics. Individuals with drug-induced lupus usually see their symptoms go away when they stop taking the medication.

Systemic lupus erythematosus (SLE) is a severe disease in which autoreactive T cells and B cells make key contributions to the pathophysiology of the disease (Wahren-Herlenius and Donner 2013, Lancet. 382:819-31; Murphy et al, 2013, Lancet. 31; 382:809-) Knockout of the PKCβ gene prevents the development of SLE in mice (Oleksyn D, et al., 2013, Arthritis Rheum 65:1022-31). This study supports the development of a selective inhibitor of PKCα, β and θ for autoimmune diseases.

### Uveitis

Uveitis is a general term describing a group of inflammatory diseases that produces swelling and destroys eye tissues. The term “uveitis” is used because the diseases often affect a part of the eye called the uvea. Nevertheless, uveitis is not limited to the uvea. These diseases also affect the lens, retina, optic nerve, and vitreous, producing reduced vision or blindness. Common symptoms of uveitis include decreased vision, pain, light sensitivity, and increased floaters.

The uvea is the middle layer of the eye which contains much of the eye's blood vessels. This is one way that inflammatory cells can enter the eye. Located between the sclera, the white outer coat of the eye, and the inner layer of the eye, called the retina, the uvea consists of the iris, ciliary body, and choroid. Uveitis disrupts vision by primarily causing problems with the lens, retina, optic nerve, and vitreous. Specific types of Uveitis, classified by where it occurs in the eye, include, anterior uveitis, intermediate uveitis, posterior uveitis, and panuveitis uveitis.

Uveitis is primarily caused by inflammatory responses inside the eye. Exemplary inflammatory responses that lead to uveitis include an attack from the body's own immune system, infections or tumors occurring within the eye or in other parts of the body, bruises to the eye, and toxins that may penetrate the eye.

Diagnosis of uveitis may include a thorough examination and the recording of the patient's complete medical history. Laboratory tests may be done to rule out an infection or an autoimmune disorder. A central nervous system evaluation is often be performed on patients with a subgroup of intermediate uveitis, called pars planitis, to determine whether they have multiple sclerosis which is often associated with pars planitis. Exemplary eye exams used, include, an eye chart or visual acuity test which measures whether a patient's vision has decreased, a funduscopic exam where the pupil is dilated with eye drops and then a light is shown through with an instrument called an ophthalmoscope to noninvasively inspect the back, inside part of the eye, measurement of ocular pressure, and a slit lamp exam which noninvasively inspects much of the eye.

Uveitis treatments primarily try to eliminate inflammation, alleviate pain, prevent further tissue damage, and restore any loss of vision. Treatments depend on the type of uveitis a patient displays. Some, such as using corticosteroid eye drops and injections around the eye or inside the eye, may exclusively target the eye whereas other treatments, such immunosuppressive agents taken by mouth, may be used when the disease is occurring in both eyes, particularly in the back of both eyes.

Steroidal anti-inflammatory medications are also often prescribed, to be taken as eye drops, swallowed as a pill, injected around or into the eye, infused into the blood intravenously, or, released into the eye via a capsule that is surgically implanted inside the eye. In order to avoid undesired side effects arising from long term use of steroids, usually other agents are started if it appears that patients need moderate or high doses of oral steroids for more than 3 months.

Other immunosuppressive agents that are commonly used include medications such as methotrexate, mycophenolate, azathioprine, and cyclosporine. In some cases, biologic response modifiers (BRM), or biologics, such as, adalimumab, infliximab, daclizumab, abatacept, and rituximab are used. These drugs target specific elements of the immune system. Some of these drugs may increase the risk of having cancer.

Treatment can also depend on the specific type of uveitis the patient is suffering from. Anterior uveitis is treated, for example, taking eye drops that dilate the pupil to prevent muscle spasms in the iris and ciliary body or taking eye drops containing steroids, such as prednisone, to reduce inflammation. Intermediate, posterior, and pan-uveitis are often treated with injections around the eye, medications given by mouth, or, in some instances, time-release capsules that are surgically implanted inside the eye.

### Encephalitis

The major role of the immune system is to recognize and fight infection. But due to dysfunction some components of the immune system may instead react with native proteins causing an autoimmune disease. When this reaction is against proteins in the brain it is termed autoimmune encephalitis (AE) and is a serious medical condition in which the immune system attacks the brain, impairing function. Autoimmune encephalitis is being increasingly recognized as important, and potentially reversible, non-infectious causes of an encephalitic syndrome. A variety autoimmune encephalitis have been described, including anti-LGI1 encephalitis (previously termed anti-voltage-gated potassium channel “anti-VGKC” antibody encephalitis) and anti-N-methyl-D-aspartic acid receptor (anti-NMDAR) encephalitis.

NMDA receptor antibody encephalitis is an autoimmune disease that causes psychiatric features, confusion, memory loss and seizures followed by a movement disorder, loss of consciousness and changes in blood pressure, heart rate and temperature. The disease can respond well to various therapies that dampen down the immune system and the removal of an underlying tumor if one is found, but improvement is often slow. The symptoms and signs seen in patients with NMDA receptor antibody associated encephalitis can be distinctive and prompt many clinicians to request the NMDA receptor antibody test to diagnose this condition. The disease mainly affects young people, with around 30% of cases under 18 years of age. Women are affected more often than men. Once a patient has been diagnosed with NMDA receptor antibody encephalitis, an underlying tumor is usually looked for. While very few males have tumors detected, recent reports suggest that between 20 and 57% of females may have an underlying tumor. The most common tumor found in women is called an ovarian teratoma. This is a non-cancerous tumor but it is thought to stimulate the production of NMDA receptor antibody.

Treatment consists of immune therapies and removal of a tumor, if present. The immune therapies use medicines to dampen down the immune system. These include steroids, immunoglobulins and plasma exchange therapies. In addition, some patients are treated with other drugs which dampen down the immune system, such as cyclophosphamide and rituximab.

When the antibodies target the voltage-gated potassium channel complex in the brain, they cause ‘Voltage-gated Potassium Channel-complex Antibody-associated Limbic Encephalitis’ (VGKC-LE). Men are roughly affected twice as often as women, with anti-LG1 antibody encephalitis. Initially, family members usually notice that their relative becomes forgetful, drowsy and withdrawn. Patients can also develop mood disorders, like depression, or bizarre thoughts and behaviors. In addition, seizures frequently occur. These may take the form of brief ‘absences’ when patients glaze over for a few seconds, also called ‘temporal lobe epilepsy’, or full blown arm and leg jerking which can be very disturbing for observers, also known as generalized seizures. Finally, patients may develop brief jerks of the face and arm, also called faciobrachial seizures. The last symptom is an important feature and highly suggestive of VGKC antibodies.

It has recently been discovered that the VGKC-antibodies do not actually target the potassium channel. They target proteins called LGI1, and less frequently CASPR2, which are tightly associated with the potassium channels in the brain. Therefore, various reports, diagnostic tests and doctors now use the terms VGKC, VGKC-complex, LGI1 and/or CASPR2 antibodies. In practice, there is usually little difference between these antibodies but this is an area currently under active research which may change the way we diagnose this disease in the future.

VGKC-LE can be treated by dampening down the immune reaction that is causing the inflammation using immunosuppression, however, no single set of medications is proven to be superior to others and research into new or optimal treatments is ongoing. Nevertheless, most clinicians opt to use immunosuppression with oral or intravenous doses of steroids intravenous immunoglobulin and/or plasma exchange therapies.

Autoimmune encephalitis may also be triggered by infection in which case the term “Post-infectious Encephalitis” is used. Acute Disseminated Encephalomyelitis (ADEM) is a Post-infectious Encephalitis. The illness usually follows in the wake of a mild viral infection, such as those that cause rashes in childhood, or immunizations. Typically there is a delay of days to two to three weeks between the triggering infection and development of the Encephalitis. ADEM accounts for around 10% of all known cases of Encephalitis. ADEM usually affects children and begins after a childhood rash, exanthema, other viral infections or immunizations. There is usually a latent period of days to two to three weeks before symptoms emerge. The illness has been poorly understood and a variety of terminologies used to describe it, these including post-viral, post-infectious or para-infectious. The illness usually begins with less-specific symptoms such as fever, headache, stiff neck, vomiting and anorexia. These are rapidly followed by depression of consciousness in which the patient may become confused and occasionally comatose. Neurological features which may be detected include visual deterioration, clumsiness in arms and legs, paralysis down one side and seizures. The duration of these symptoms is variable. Some cases last a few weeks to a month, while other fatal cases have a rapid progressive course over a number of days.

There is a general agreement that a causative organism cannot be isolated from the brain of patients with ADEM. The association of the disease with a previous infection or immunization suggests an immunological process. Detailed laboratory studies involving measurement of anti-brain antibodies and of cellular immune responses to specific brain antigens suggest that these patients have developed an allergic response against their own brain constituents and this is an ‘autoimmune’ response.

The ideal form of treatment is immunomodulation which should be instituted once the diagnosis is made and has more benefit when given early. The diagnosis, however, may be difficult to make swiftly. High doses of steroids can often lead to a rapid resolution of symptoms with an excellent prognosis.

### Rheumatoid Arthritis

Rheumatoid arthritis (RA) is a chronic autoimmune disorder in which the body's immune system attacks the joints and additional organs such as skin, eyes, lungs, and blood vessels. In some instances, symptoms of RA include pain, swollen and/or stiffness of the joints, rheumatoid nodules, low red blood cells, and inflammation around the lungs and heart.

In some instances, RA is further classified into rheumatoid factor positive (seropositive) RA, rheumatoid factor negative (seronegative) RA, and juvenile RA (orjuvenile idiopathic arthritis). Rheumatoid factor (RF) is an autoantibody directed against the Fc region of IgG. In some cases, rheumatoid factor comprises one or more isotype of immunoglobulin, such as for example, IgA, IgG, IgM, IgE, or IgD. In some cases, rheumatoid factor also includes a cryoglobulin, an antibody that precipitates at temperatures below normal body temperature. Presence or absence of rheumatoid factor (i.e., seropositive or seronegative) is used as part of a diagnostic tool in evaluating the presence and progression of RA. Juvenile RA affects children under age 16 in which the inflammation duration last more than 6 weeks.

In some embodiments, both Th17 and Th1 have been implicated in the development and progression of RA. For example, overexpression of IL-17 by Th17 cells leads to synovial inflammation, cartilage destruction, and bone erosion. Furthermore, IL-17 triggers human synoviocytes to produce IL-6, IL-8 GM-CSF, and PGE2, and triggers the production of TNF-α, IL-1β, IL-12, stromelysin, IL-10, and IL-1R antagonist in human peripheral blood macrophages. In some instances, Th17 cells have also been observed to coexpress the Th1 cytokine IFN-γ in peripheral blood, suggesting a plasticity of Th17 cells given rise to Th1 cells. (Nistala, et al., “Th17 plasticity in human autoimmune arthritis is driven by the inflammatory environment,” PNAS 107(33):14751-14756 (2010)).

In some cases, PKC (e.g., PKC-0) has been implicated in mounting a Th-1 dependent response. Indeed, PKC-θ-deficient mice exhibit decreased disease severity in both mBSA-induced arthritis and collagen-induced arthritis (CIA) mouse models, reduced proliferative capacity of PKC-θ-deficient T cells in response to Ag and decreased IL-2 levels, impaired expression of T-bet, and reduced levels of IFN-γ and IL-4. Furthermore, PKC-θ deficiency correlates to a reduced T cell proliferation, Th1/Th2 cell differentiation, and T cell activation before and during disease peak. (Healy, et al., “PKC-θ-deficient mice are protected from Th1-dependent antigen-induced arthritis,” J Immunol 177:1886-1893 (2006)).

In some embodiments, treatment of RA include disease-modifying antirheumatic drugs (DMARDs) such as methotrexate, hydroxychloroquine, sulfasalazine, leflunomide, abatacept, or anakinra; biologics such as tumor necrosis factor alpha blockers (e.g., infliximab), interleukin 1 blockers (e.g., anakinra), monoclonal antibodies (e.g., rituximab, tocilizumab), T cell costimulation blockers (e.g., abatacept); nonsteroidal anti-inflammatory drugs (NSAIDs); COX-2 inhibitors (e., celecoxib); glucocorticoids; or surgery.

### Multiple Sclerosis

Multiple Sclerosis (MS), also known as disseminated sclerosis or encephalomyelitis disseminata, is a demyelinating disease in which the myelin sheath of neurons, or the fatty sheath that surrounds and insulates nerve fibers in the brain and spinal cord, is damaged. In some instances, symptoms of MS include numbness or weakness of one or more portions of the body, partial or complete loss of vision, prolonged double vision, tingling or pain, Lhermitte sign, tremor, slurred speech, fatigue, dizziness, and impaired bowel and bladder functions.

In some embodiments, there are several phenotypes or disease course associated with MS. In some instances, these include relapsing-remitting (RR), secondary progressive (SPMS), primary progressive (PPMS), progressive relapsing, clinically isolated syndrome (CIS), and radiologically isolated syndrome (RIS). In some cases, the relapsing-remitting subtype begins with a clinically isolated syndrome (CIS). CIS is an attack suggestive of demyelination but does not fulfill the criteria for MS. Secondary progressive (SP) MS is characterized by a progressive neurologic decline between acute attacks without a definite period of remission. In some instances, about 65% of those with relapsing-remitting MS progresses into SPMS. Primary progressive (PP) MS is characterized by progression of disability from onset, with no or occasional and minor remissions and improvements. Progressive relapsing MS is characterized by a steady neurologic decline with clear superimposed attacks.

In some embodiments, both B cells and T cells play a role in the development and progression of MS. For example, deregulation of pro-inflammatory cytokines such as Th1 cytokine IFNγ leads to a disruption of the blood brain barrier (BBB) (Compston, A. and Coles, A. “Multiple sclerosis,” Lancet 372:1502-1517 (2008)). Furthermore, secretion of IL-17 and IL-22 by Th17 cells increases permeability of the BBB by disruption of the endothelial tight junction and by interaction with endothelium to allow further recruitment of CD4+ subsets (Hoglund, R. A., and Maghazachi, A. A. “Multiple sclerosis and the role of immune cells,” World J Exp Med. 4(3):27-37 (2014)). As such, the presence of pro-inflammatory cytokines leads to complement deposition and opsonization of the surrounding tissues of the perivascular space and parenchyma, local activation of microglia and macrophages causing demyelination, and neuronal cell death (Prineas, J. W., and Graham, J. S. “Multiple sclerosis: capping of surface immunoglobulin G on macrophages engaged in myelin breakdown.” Ann Neurol. 10:149-158 (1981)). In some instances, B cells further contribute to the pathology of MS through antigen presentation, cell interactions and/or production of immunoglobulins from plasma cells (Hestvik, A. L. “The double-edged sword of autoimmunity: lessons from multiple sclerosis,” Toxins 2:856-877 (2010)).

In some instances, T cell activation requires T cell receptor (TCR) interaction with MHC-peptide complexes in parallel with engagement of costimulatory molecules such as CD28. In some cases, PKC-θ is associated with TCR- and CD28-specific signals leading to T cell activation, proliferation, and cytokine production. Indeed, a study has shown that PKC-θ is important for the development of Ag-specific Th1 cells in experimental allergic encephalomyelitis (EAE), a mouse model of MS (Salek-Ardakani, et al., “Protein kinase CO controls Th1 cells in experimental autoimmune encephalomyelitis,” J Immunol 175:7635-7641 (2005)).

PKC theta is involved in modulating both Th1 and Th2 type responses. For example, in a MOG-induced EAE model of MS, a Th1-based model, mice deficient in PKC theta were protected from disease development. Furthermore, Th-1 cytokines such as IL-2 and IFNγ were observed to decrease in the absence of PKC theta. (Anderson, et al., “Mice deficient in PKC theta demonstrate impaired in vivo T cell activation and protection from T cell-mediated inflammatory diseases,” Autoimmunity, 39(6): 469-478 (2006))

PKC-θ is involved in the regulation of multiple T cell functions that are necessary for the development of autoimmune diseases. PKC-θ ablation leads to reduced production of Th1 cytokine IFNγ but not IL-2 or IL-4, and reduced production of T cell effector cytokine IL-17. PKC-θ ablation further fails to up-regulate LFA-1 expression in response to TCR activation, which is responsible for T cell transendothelial adhesion, and in some instances LFA-1 upregulation is involved in the induction of EAE. (Tan, et al., “Resistance to experimental autoimmune encephalomyelitis and impaired IL-17 production in protein kinase C {theta}-deficient mice,” J. Immunol. 176: 2872-2879 (2006)).

PKC-θ is important for the development and persistence of Ag-specific Th1 cells in EAE. A PKC-θ deficiency affects the peripheral T cell responses of mice to MOG, leading to diminished inflammatory cells in CNS tissue and a lowering of Th1 cytokine production, resulting in delayed EAE onset and minimal clinical signs of disease. (Salek-Ardakani, et al., “Protein kinase C{theta}controls Th1 cells in experimental autoimmune encephalomyelitis,” J. Immunol. 175: 7635-7641 (2005)).

### Inflammatory Bowel Disease

Inflammatory bowel disease (IBD) is a group of inflammatory conditions of the digestive tract. In some instances, IBD is further classified into Crohn's disease, ulcerative colitis, collagenous colitis, lymphocytic colitis, diversion colitis, Behçet's disease, and indeterminate colitis.

Crohn's disease, also known as Crohn syndrome or regional enteritis, is an IBD that affects the gastrointestinal tract. Symptoms of Crohn's disease include abdominal pain, diarrhea, fever, and weight loss. Additional complications include anemia, skin rashes, arthritis, inflammation of the eye, and tiredness. Although the exact cause is unknown, in some instances, a combination of environmental factors, immune and bacterial factors, and genetic predisposition has been implicated in the development of this disease. In some instances, treatment include antibiotics, 5-aminosalicylic acid (5-ASA) drugs, corticosteroids such as prednisone, immunomodulators such as azathioprine and methotrexate, biologics such as infliximab, adalimumab, certolizumab, and natalizumab, and surgery.

Ulcerative colitis (UC, or Colitis ulcerosa) is a form of IBD that causes inflammation and ulcers in the colon. The symptom of ulcerative colitis include diarrhea which in some instances is mixed with blood and mucus, weight loss, abdominal pain, and anemia. In some instances, treatment include 5-aminosalicylic acid (5-ASA) drugs such as sulfasalazine and mesalazine, corticosteroids such as prednisone, immunosuppressive medications such as azathioprine, and biologics such as infliximab, adalimumab, and golimumab.

### Optic Neuritis

Optic neuritis is inflammation of the optic nerve. It is further classified into papillitis and retrobulbar neuritis. Papillitis is characterized by inflammation of the optic nerve head, and retrobulbar neuritis is characterized by inflammation of the posterior of the nerve. In some instances, multiple sclerosis is one of the most common etiology of optic neuritis. Additional causes include infection (e.g. syphilis, Lyme disease, herpes zoster), autoimmune disorders (e.g. lupus, neurosarcoidosis, neuromyelitis optica), inflammatory bowel disease, drug induced (e.g. chloramphenicol, ethambutol, isoniazid, streptomycin, quinine, penicillamine, amino salicylic acid, phenothiazine, phenylbutazone), vasculitis, B12 deficiency and diabetes. The symptoms of optic neuritis include sudden blurred or foggy vision, pain associated with eye movement, impaired color vision, and impaired depth perception. In some instances, treatment includes corticosteroids.

### Neuromyelitis Optica

Neuromyelitis optica (also known as Devic's disease, Devic's syndrome, or NMO) is a B-cell mediated disease associated with simultaneous inflammation and demyelination of the optic nerve (optic neuritis) and the spinal cord (myelitis). In some instances, the symptoms include vision loss, pain sensation within the eye, sensory disturbances, weakness, numbness and/or paralysis of the arms and legs, and loss of bladder and bowel control. In the disease process, autoantibodies NMO-IgG, derived from peripheral B cells, target CNS astrocytic Aquaporin 4 (AQP4), resulting in complement activation and inflammation. In some instances, the inflammatory lesions are similar to the lesions of multiple sclerosis (MS); however, they differ from MS in their perivascular distribution. There are two variants of neuromyelitis optica, AQP4+ NMO which leads to the attack of astrocytes of the optic nerves and spinal cords by a person's own immune system, and AQP4- NMO, in which the etiology is unknown.

In some embodiments, neuromyelitis optica belongs to a collection of similar diseases termed neuromyelitis optica spectrum disorder (NMOSD). In some cases, the additional diseases belonging to NMOSD comprise Standard Devic's disease, limited forms of Devic's disease, Asian optic-spinal MS, longitudinally extensive myelitis or optic neuritis associated with systemic autoimmune disease, optic neuritis, or NMO-IgG negative NMO.

### Sjögren's Syndrome

Sjögren's syndrome is a chronic autoimmune disease in which the exocrine glands such as the salivary and lacrimal glands are destroyed by the leukocytes or the white blood cells. In some instances, skin and organs such as kidneys, blood vessels, lungs, liver, biliary system, pancreas, peripheral nervous systems, and the brain are also affected. In some cases, Sjögren's syndrome is classified as primary or secondary Sjögren's syndrome. Symptoms include xerostamia (i.e. dry mouth), keratoconjunctivitis sicca (i.e. dry eyes), joint pain, swollen salivary glands, skin rashes or dry skin, vaginal dryness, persistent dry cough, and prolonged fatigue. In some cases, treatments include parasympathomimetic agonists such as cevimeline and pilocarpine, nonsteroidal anti-inflammatory drugs (NSAIDs), immunosuppressant such as methotrexate, hydroxychloroquine, or surgery.

### Psoriasis

Psoriasis is an autoimmune disease characterized by regions of abnormal skin. In some instances, psoriasis is further classified into plaque, guttate, inverse, pustular, and erythrodermic. Plaque psoriasis or psoriasis vulgaris comprise about 90% of total cases. It is characterized by the presence of red patches with white scales on top. In some cases, plaque psoriasis occurs at the forearms, shins, navel, and the scalp region. Guttate psoriasis is characterized by drop shaped lesions. Pustular psoriasis is characterized by small non-infectious pus filled blisters. Inverse psoriasis is characterized by red patches in the skin fold regions. Erythrodermic psoriasis is characterized by rashes throughout the body and in some instances further develops into the subtypes of psoriasis. In some instances, psoriasis in combination with inflammation of the joints is terms psoriatic arthritis. In some embodiments, treatments of psoriasis include nonsteroidal anti-inflammatory drugs (NSAIDs); immunosuppressant such as methotrexate; fumarates such as dimethyl fumarate; biologics such as infliximab, adalimumab, golimumab, and certolizumab pegol; retinoids; vitamin D3 cream, or phototherapy such as ultraviolet light.

### Systemic Scleroderma

Systemic scleroderma, also known as systemic sclerosis or SSc, is a connective tissue disease characterized by sclerosis or hardening of skin, blood vessels, and internal organs, and inflammation of joints and muscles. In some instances, systemic scleroderma is further classified into limited cutaneous scleroderma (lcSSc), diffuse cutaneous scleroderma (dcSSc), and systemic sclerosis sine scleroderma (ssSSc). Limited cutaneous scleroderma affects the face, hands and feet, and is characterized by calcinosis, Raynaud phenomenon, esophageal dysfunction, sclerodactyly, and telangiectasia. Diffuse cutaneous scleroderma affects the skins throughout the body and in some instances progress to visceral organs such as the kidneys, heart, lungs and gastrointestinal tract. Systemic sclerosis sine scleroderma is characterized by organ fibrosis in the absence of cutaneous sclerosis. In some cases, treatments include calcium channel blockers, prostanoids, tadalafil, bosentan, corticosteroids, and immunosuppressant.

### Alkylosing Spondylitis

Alkylosing spondylitis (also known as Bekhterev's disease, Marie-Strumpell disease, or AS) is a chronic inflammatory disease of the axial skeleton. Alkylosing spondylitis mainly affects the spinal joints and the sacroiliac joint of the pelvis, although in some instances peripheral joints and nonarticular structures are also involved. In some cases, alkylosing spondylitis is characterized by the ossification of the outer fibers of the fibrous ring of the intervertebral discs, and in severe cases with complete fusion of the spine. Symptoms of alkylosing spondylitis include pain and stiffness of lower back and hips, gradual loss of spinal mobility and chest expansion, limitation of anterior flexion, lateral flexion, and extension of the lumbar spine. In some instances, treatments include nonsteroidal anti-inflammatory drugs (NSAIDs) such as ibuprofen, phenylbutazone, diclofenac, indomethacin, naproxen and COX-2 inhibitors; opioid analgesics, disease-modifying antirheumatic drugs (DMARDs) such as sulfasalazine; tumor necrosis factor-alpha blockers such as etanercept, infliximab, golimumab, and adalimumab; anti-interleukin-6 inhibitors such as tocilizumab and rituximab.

### Autoimmune Hepatitis

Autoimmune hepatitis (AIH) or lupoid hepatitis is characterized by chronic inflammation of the liver. In some instances, symptoms include fatigue, muscle aches, fever, jaundice, and upper right quadrant abdominal pain. In some cases, autoimmune hepatitis is further classified into four subtypes: positive antinuclear antibody (ANA) and anti-smooth muscle antibody (SMA), characterized by elevated immunoglobulin G; positive liver/kidney microsomal antibody (LKM-1, LKM-2, or LKM-3); positive antibodies against soluble liver antigen; and no autoantibodies detected. In some cases, treatments include glucocorticoids such as budesonide and prednisone; and immunosuppressant such as azathioprine, mycophenolate, cyclosporin, tacrolimus, methotrexate, and the like.

PKC-θ modulates the activation ofNKT cells to induce hepatitis. For example, mice deficient in PKC-θ were resistant to concanavalin A (ConA)-induced hepatitis and that ConA-induced production of cytokines such as IFNγ, IL-6, and TNFα, which mediate the inflammation responsible for liver injury, were lower in PKC-θ deficient mice. (Fang, et al., “Ameliorated ConA-induced hepatitis in the absence of PKC-theta,” PLoS ONE, 7(2): e31174 (2012)).

### Allogeneic Conditions

### Organ Transplant Rejection

Organ transplant rejections occur when the transplanted tissue is rejected by the host's immune system. In some instances, the transplanted organs include solid organs such as heart, lungs, kidneys, liver, stomach, pancreas, or intestine, or tissues derived from solid organs such as skin, heart valves, veins, or corneas. In some cases, organ transplant rejection is characterized by hyperacute rejection, acute rejection and chronic rejection. Hyperacute rejection occurs when the transplanted tissue is rejected within minutes or hours due to vascularization damage. Acute rejection occurs within the first six months after transplantation, and further comprises acute cellular rejection and humoral rejection. Chronic rejection occurs after six month of transplantation.

In some instances, alloreactivity in transplantation arises when a mismatch of donor-host human leukocyte antigen (HLA) occurs, leading to subsequent B-cell and T-cell mediated responses. For example, in a B-cell mediated response, allogeneic HLA antigens are internalized by B cells and subsequently processed into peptides for presentation on HLA class-II molecules. Recognition of the HLA class-II presented HLA-derived epitopes by CD4+ T cells results in B-cell activation and IgM to IgG isotype switching. As such, donor-specific IgG HLA alloantibodies are produced which recognize the allogeneic HLA molecules, leading to rejection of the transplanted organ. In a T-cell mediated response, alloreactive T cells either directly recognize intact allogeneic HLA molecules or are involved in indirect recognition by modulating B-cell activation and IgG isotype switching.

In some instances, PKC (e.g., PKC-θ, PKC-α) is involved in survival of activated T cells. Indeed, a study has shown that injection of allogeneic cells into a PKC-θ deficient mice provoked a decreased T cell response compared to WT mice and that alloreactive T cells undergo apoptosis in the absence of PKC-θ. (Sun, Z. “Intervention of PKC-θ as an immunosuppressive regimen,” Frontiers in Immunology 3(225):1-9 (2012); Anderson, et al., “Mice deficient in PKC theta demonstrate impaired in vivo T cell activation and protection from T cell-mediated inflammatory diseases,” Autoimmunity 39:469-478 (2006); Manicassamy, et al., “Protein kinase C-{theta}-mediated signals Enhance CD4+ T cell survival by up-regulating Bcl-xL,” J. Immunol. 176:6709-6716 (2006)) A second study shows that a combination of PKC-θ/PKC-α deficiency leads to an additive T cell response defects (Gruber, et al., “PKCθ cooperates with PKCα in alloimmune responses of T cells in vivo,” Molecular Immunology 46:2071-2079 (2009)).

In some embodiments, there are several different treatment options for acute rejection. Exemplary treatment options include corticosteroids such as prednisolone and hydrocortisone; calcineurin inhibitors such as cyclosporin and tacrolimus; anti-proliferatives such as azathioprine and mycophenolic acid; mTOR inhibitors such as sirolimus and everolimus; biologics such as monoclonal anti-IL-2Rα receptor antibodies (e.g., basiliximab, daclizumab), polyclonal anti-T-cell antibodies (e.g., anti-thymocyte globulin and anti-lymphocyte globulin), and monoclonal anti-CD20 antibodies (e.g., rituximab). For hyperacute rejection, the sole treatment option is removal of the tissue, and for chronic rejection, retransplant is proposed as the preferred option.

PKC-θ enhances T cell survival and promotes the differentiation of naive T cells into inflammatory Th17 cells. Furthermore, modulation of PKC-θ activity shifts the ratio between inflammatory effector T cells and inhibitory Tregs, to control T cell-mediated immune responses that are responsible for autoimmunity and allograft rejection. Indeed, PKC-θ-deficient mice are resistant to the development of several Th2 and Th17-dependent autoimmune diseases and are defective in mounting alloimmune responses required for rejection of transplanted allografts and graft-versus-host disease. (Sun, Z. “Intervention of PKC-θ as an immunosuppressive regiment,” Frontiers in Immunology, 3(225):1-9 (2012))

### Graft Vs Host Disease

Graft vs host disease (GvHD) is a complication following an allogeneic stem cell transplant, and is characterized by a T cell-mediated recognition of minor histocompatibility antigens followed by organ-specific vascular proliferation, cytokine release, and direct cell-mediated attack on normal tissues. In some cases, the stem cells are obtained from bone marrow, peripheral blood, or cord blood. In some instances, there are two types of GvHD, acute or fulminant form of GvHD (aGvHD), and chronic form of GvHD (cGvHD). Acute GvHD occurs within the first 100 days of transplant while chronic GvHD occurs after the 100 day time frame. In some instances, treatment of GvHD include calcineurin inhibitors such as cyclosporine and tacrolimus; mTOR inhibitors such as sirolimus; and antiproliferative agents such as methotrexate, cyclophosphamide, and mycophenolate.

PKC-θ plays an important role in lowering the overall signaling threshold required for T cell activation. Therefore, the absence of PKC-θ selectively impairs T cell activation by low-level and low-affinity TCR agonists. As such, in an allogeneic setting, inhibition of PKC-θ can prevent GVHD induction while maintaining the ability to respond to virus infection and to induce graft-versus-leukemia (GVL) effect after BM transplantation. (Valenzuela, et al., “PKCθ is required for alloreactivity and GVHD but not for immune responses toward leukemia and infection in mice,” The Journal of Clinical Investigation, 119(12): 3774-3786 (2009)).

3. Inflammation

PKCβ also play a role in inflammation (as indicated in the sections above), such as inflammation caused by inflammatory bowel disease is Crohn's disease, ulcerative colitis, collagenous colitis, lymphocytic colitis, diversion colitis, Behçet's disease, or indeterminate colitis.

### Dosing

The dose when using the compositions of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis conducted or on whether further active compounds are administered in addition to the pharmaceutical composition as disclosed herein. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g, into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The pharmaceutical preparations are preferably in unit dosage forms. In such form, the preparation is subdivided into unit doses containing appropriate quantities ofthe active component. The unit dosage form can be a packaged preparation, the package containing discrete quantities of preparation.

### Combination Therapy

In some embodiments, the PKCβ inhibitor disclosed herein is administered in combination with at least one additional pharmaceutical agent. Administration of the PKCβ inhibitor and the additional pharmaceutical agent can occur simultaneously or sequentially by the same or different routes of administration.

In some embodiments, the at least one additional pharmaceutical agent is administered to the patient prior to initiation of the administration of the PKCβ inhibitor. In some embodiments, the at least one additional pharmaceutical agent is administered for at least one week, or at least two weeks, or at least three weeks, or at least one month, or at least two months, or at least three months prior to initiation of the administration of the PKCβ inhibitor.

The suitability of a particular route of administration employed for a particular pharmaceutical agent will depend on the pharmaceutical agent itself (e.g., whether it can be administered orally or topically without decomposition prior to entering the blood stream) and the subject being treated. Particular routes of administration for the additional pharmaceutical agents or ingredients are known to those of ordinary skill in the art.

The amount of additional pharmaceutical agent administered can be determined based on the specific agent used, the subject being treated, the severity and stage of disease and the amount(s) of the at least one PKCβ inhibitor and any optional additional known pharmaceutical agents concurrently administered to the patient. The at least one additional pharmaceutical agent, when employed in combination with at least one PKCβ inhibitor, may be used, for example, in those amounts indicated in the Physicians' Desk Reference (PDR) or as otherwise determined by one of ordinary skill in the art.

In some embodiments, the dose of the at least one additional pharmaceutical agent is reduced when used in combination with the at least one PKCβ inhibitor. In some embodiments, the dose is not reduced.

Some embodiments of the present invention include a method of producing a pharmaceutical composition for “combination therapy” comprising admixing at least one PKC□ inhibitor together with at least one additional pharmaceutical agent as described herein and a pharmaceutically acceptable carrier.

In some instances, the methods described herein further comprise combination therapy with at least one additional oncology therapeutic agent. In some embodiments, the additional oncology therapeutic agent is selected from a SYK inhibitor, a dual SYK-JAK inhibitor, a PI3K inhibitor, a JAK-STAT inhibitor, a BCL2 inhibitor, an immunomodulatory agent, an antibody-drug conjugate, an immune checkpoint inhibitor, a PD-1 inhibitor, a TIM-3 inhibitor, a CTLA-4 inhibitor, a bromodomain inhibitor, an EZH2 inhibitor, an HDAC inhibitor, or an IDH2 inhibitor.

### BTK Inhibitors

In some embodiments, the additional oncology therapeutic agent is a BTK inhibitor.

The Bruton's tyrosine kinase (BTK) inhibitor ibrutinib is an FDA approved anticancer drug targeting B-cell malignancies. Other BTK inhibitors currently in some stage of clinical development include, but are not limited to: ONO/GS-4059 (Ono Pharmaceuticals/Gilead Sciences), AVL-292/CC-292/spebrutinib (Celgene Corporation), BGB-3111 (BeiGene), and ACP-196/acalabrutinib (Acerta Pharma), M7583 (EMD Serono/Merck KGaA), MSC2364447C(EMD Serono/Merck KGaA), BIIB068 (Biogen), ACO0058TA (ACEA Biosciences), and DTRMWXHS-12 (Zhejiang DTRM Biopharma).

### Hydrates and Solvates

The term “hydrate” as used herein means a compound or a salt thereof, that further includes a stoichiometric or non-stoichiometric amount of water bound by non-covalent intermolecular forces. The term “solvate” as used herein means a compound or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to salts described herein, it embraces pharmaceutically acceptable solvates and/or hydrates of such salts.

It will be apparent to those skilled in the art that the compositions described herein may comprise, as the active component, either a compound described herein or a pharmaceutically acceptable salt or as a pharmaceutically acceptable solvate or hydrate thereof. Moreover, various hydrates and solvates of the compounds described herein and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc, New York, 1999. Accordingly, one aspect of the present invention is directed to methods of administering pharmaceutical composition comprising hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

### Other Utilities

Other uses of the disclosed compositions will become apparent to those skilled in the art based upon, inter alia, a review of this disclosure.

As will be recognized, the steps of the methods of the present invention need not be performed any particular number of times or in any particular sequence. Additional objects, advantages and novel features of this invention will become apparent to those skilled in the art upon examination of the following examples thereof, which are intended to be illustrative and not intended to be limiting.

### EXAMPLES

The pharmaceutical compositions disclosed herein and their preparation are further illustrated by the following examples. The following examples are provided to further define the invention without, however, limiting the invention to the particulars of these examples. The compounds described herein, supra and infra, are named according to the CS ChemDraw Ultra Version 7.0.1, AutoNom version 2.2, CS ChemDraw Ultra Version 9.0.7, or CS ChemDraw Ultra Version 12.0. In certain instances, common names are used and it is understood that these common names would be recognized by those skilled in the art.

### Example 1. PKCβ Signaling Assay

Previous work has demonstrated that stimulation with phorbol myristic acid (PMA) and ionomycin stimulates PKCβ. This has been shown to lead to PKCβ-mediated phosphorylation of the PAI-1 mRNA binding protein SERBP1 (O'Brien (2014) Cancer Res 73, 3195-3195). To monitor PKCβ mediated signaling, a biomarker marker assay was developed using Alexa-647 label anti-phospho_SERBP1 antibody.

### Sample Acquisition

Briefly, whole blood specimens from each patient was drawn into sodium citrate collection tubes. Samples will be shipped overnight to the flow cytometry lab for processing and testing

Duplicates will be prepared for each treatment condition per donor. The following conditions will be prepared for each donor, unstimulated and PMA and ionomycin stimulated samples. Whole blood 200 μL will be placed in appropriate size cryovial. The samples will be treated as described in Table 1 and incubated at 37° C., 5.0% CO2 for 25-30 minutes.

TABLE 1
Stimulation

| | | # of |
|---|---|---|
| Condition | Stimulants | Replicates |
| Unstimulated | 0 | 2 |
| PMA + | PMA 200 nM (4.94 μL of | 2 |
| ionomycin | 8.1 μM*) + 1 μg/mL | |
| | Iono (2 μL\hat{ }) | |

*Prepare PMA 8.1 μM by adding 1 μL of 8.1 mM (5 mg/mL) and 999 μL RPMI 1640 media containing 10% FBS
\hat{ }100 μg/mL Ionomycin prepared by adding 20 μL of 1 mg/ml and 180 μL RPMI 1640 media containing 10% FBS

Following stimulation 2 mL 1×BD FACSLyse will be added, then vortexed and incubated for 12-15 minutes in the dark at ART. Samples will be stored immediately at −80° C. until testing.

Samples from each subject will be processed in batches with all timepoints and include a normal human control unstimulated and stimulated sample. All samples will be processed and collected in singlet.

Frozen samples will be thawed by placing samples in a 37° C. water or bead bath. Sample will be transferred to tubes and centrifuged at 1700 rpm, for 5 minutes at ambient room temperature (ART) with Brake on. Samples will be decanted and washed twice with 1 mL Stain Buffer (FBS). Samples will be resuspended in 200 μL Stain Buffer (FBS) and 100 μL transferred to appropriate volume of fluorochrome-conjugated monoclonal surface antibodies will be added (Table 2).

TABLE 2
Surface Antibody Panel Whole Blood

| FITC | PE | AF647 | PECy7 | APCCy7 | V421 | V510 |
|---|---|---|---|---|---|---|
| HLADR | CD86 | — | CD20 | CD19 | CD45 | CD3 |
| (5 μL) | (5 μL) | | (5 μL) | (5 μL) | (5 μL) | (5 μL) |

Samples will be hand mixed and incubated for 15-20 minutes in the dark at ART. Samples will be washed twice with 1 mL Stain Buffer (FBS). After wash, 200 μL Fix/Perm buffer will be added to each sample, then vortexed and incubated for 30-35 minutes in the dark at 2-8° C. Samples will be washed twice with 1 ml 1× Permeabilization Buffer (Perm Buffer). 100 μL of 1× Perm Buffer and appropriate volume of pSERBP1 AF647 will be added (documented in the study paperwork) and incubated for 30-35 minutes in the dark at 2-8° C. Samples will be washed twice with 1 mL 1× Perm Buffer. Samples will be resuspended in 125 μL 1× Stain Buffer FBS for acquisition on the flow cytometer.

### Flow Cytometer Calibration

For the flow cytometer, routine fluidics and calibration check will be performed on each day of testing by running BD Cytometer Set-up and Tracking Beads and Spherotech Ultra Rainbow Beads per SOP. Fluorescence compensation to address potential spill-over of one fluorescence signal into another will also be conducted at the time of initial instrument. Additional electronic compensation may be performed if necessary after data acquisition using FACSDiva™ software (version 6.1.3 or higher).

### Acquisition and Analysis of Samples

Flow cytometric data acquisition will be performed using the BD FACSCantoII™ which evaluates two scatter parameters and up to eight color fluorescence channels. Data will be acquired using BD FACSDiva™ software (version 6.1.3 or higher). The samples will be acquired to distinguish the cells of interest from other cell types in the peripheral blood by electronic gating on the basis of CD45 versus side scatter. The instrument will be set to collect 50,000 CD45+ lymphocyte events. Dual combination cytograms and/or histograms will be generated to illustrate the LL (−/−), LR (+/−), UL (−/+), UR (+/+) and/or interval gate for the fraction of the cells. The Flow Cytograms will be printed and maintained with study binder.

The Median Fluorescence Intensity for the AF647 channel will be reported for each population (CD45, CD3, CD19 and CD20). Relative % data will also be reported for CD3+, CD3−CD19+, CD3−CD20+, CD3−CD19+HLADR+CD86+, and CD3−CD20+HLADR+CD86+. Data will be analyzed by Microsoft Excel to obtain descriptive statistics; i.e. Mean, SD, and CV %.

TABLE 3
Gating Strategy

| Cytogram/ | | | | |
|---|---|---|---|---|
| Histogram | X-axis | Y-axis | Gated on | Population |
| Cytogram 1 | FSC | SSC | No gate | P1 = Total cells |
| Cytogram 2 | CD45 V421 | SSC | P1 | P2 = Lymphocytes |
| Cytogram 3 | CD3 V510 | SSC | P2 | P3 = CD3+ cells |
| Cytogram 4 | CD3 V510 | CD19 APCCy7 | P2 | P4 = CD3-CD19+ cells |
| Cytogram 5 | CD3 V510 | CD20 PECy7 | P2 | P5 = CD3-CD20+ cells |
| Cytogram 6 | HLADR FITC | CD86 PE | P4 | Quadrant marker |
| Cytogram 7 | HLADR FITC | CD86 PE | P5 | Quadrant marker |
| Histogram 1 | pSERBP1 AF647 | Count | P2 | MFI AF647 |
| Histogram 2 | pSERBP1 AF647 | Count | P3 | MFI AF647 |
| Histogram 3 | pSERBP1 AF647 | Count | P4 | MFI AF647 |
| Histogram 4 | pSERBP1 AF647 | Count | P5 | MFI AF647 |

### Data Analysis

The flow cytometry data are analyzed to determine the amount of SERBP1 phosphorylation following PMA/ionomycin stimulation. The reported data are the CD19+pSERBP1+ population, normalized to each patient's own unstimulated sample at the corresponding time point or an unstimulated sample collected from and individual prior to exposure to Compound A.

Biomarker data from a PKCβ signaling assay performed on whole blood samples from patients with CLL or SLL suggests that concentrations of Compound A in the plasma in the range of 500-600 ng/mL completely suppress PKCβ signaling.

TABLE 4
Inhibition of PKCβ signaling in β-cells after PMA stimulation
vs. Compound A Drug Concentration

| | | | Mean | | |
|---|---|---|---|---|---|
| | | Mean | Stimulation | % Inhibition | Compound A |
| Number | Dose | Stimulation | at Day 8 | of | Plasma |
| of | (mg | by PMA | 3 h Post- | Stimulation | Concentration |
| Patients | BID) | [No drug] | Dose Peak | at Peak | (ng/mL) |
| N = 1 | 100 | 119% | 96% | 122% | 587 |
| N = 3 | 200 | 171% | 100% | 102% | 837 |
| N = 3 | 250 | 239% | 102% | 98% | 1310 |
| | | | Mean | | |
| | | | Stimulation | % Inhibition | |
| | | Mean | at Day 8 | of | Compound A |
| Number | Dose | Stimulation | Pre-Dose | Stimulation | Plasma |
| of | (mg | by PMA | Trough | at Trough | Concentration |
| Patients | BID) | [No drug] | (Cₘᵢₙ) | (Cₘᵢₙ) | (ng/mL) |
| N = 1 | 100 | 119% | 109% | 51% | 186 |
| N = 3 | 200 | 171% | 121% | 70% | 295 |
| N = 3 | 250 | 239% | 104% | 97% | 542 |

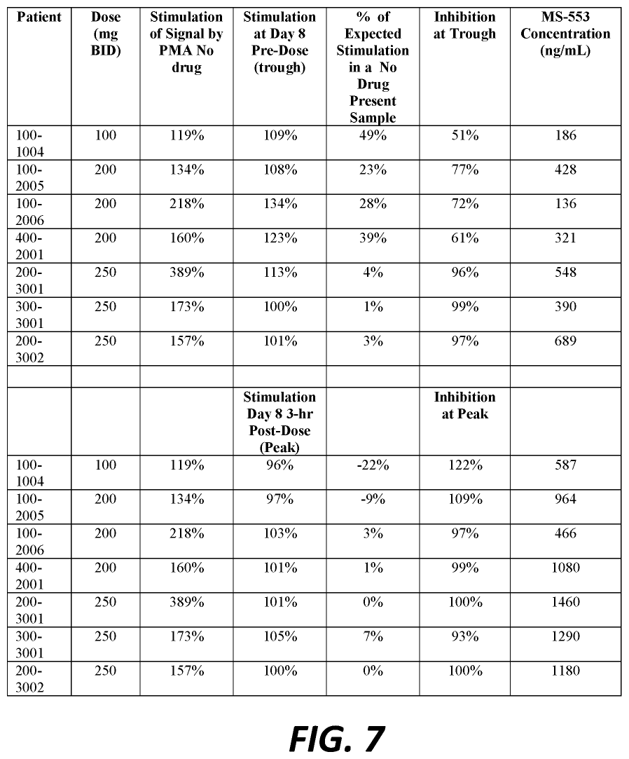

FIG. 7 shows the individual patient data for this example.

### Example 2: Preparation of Tablets Containing Modified Release Compositions

A desirable feature of a modified-release composition is a stable release profile, i.e, in which the release rate of the drug does not vary substantially over time. For example, a desirable feature is a release rate that does not vary substantially over a time period during which the drug is in storage. Accordingly, various combinations of excipients are tested with the goal of obtaining a composition having a release rate of Compound A that would be stable over time.

An example of a suitable composition is a composition in the form of a tablet. Examples of modified-release tablets at various doses of Compound A are shown in Table 5A-C.

TABLE 5A

| Ingredient | % (w/w) | mg/tablet | % (w/w) | mg/tablet | % (w/w) | mg/tablet |
|---|---|---|---|---|---|---|
| Compound A | 37.5 | 300.0 | 36 | 200.2 | 30 | 150 |
| HydroxyPropyl Cellulose | 4.0 | 32.0 | 5 | 27.8 | 4 | 20 |
| (HPC-Klucel EXF) | | | | | | |
| PolyOx N60K | 25.0 | 200.0 | 25 | 139.0 | 25 | 125 |
| Mannitol (Mannogem XL SD) | 16.3 | 130.0 | 16.5 | 91.7 | 18.65 | 93.25 |
| Dicalcium Phosphate | 16.3 | 130.0 | 16.5 | 91.7 | 21.35 | 106.75 |
| (Emcompress) | | | | | | |
| Magnesium stearate | 1.0 | 8.0 | 1 | 5.6 | 1 | 5 |
| Total | 100 | 800.0 | 100 | 556.0 | 100 | 500 |

TABLE 5B

| | | mg/ | % | mg/ | % | mg/ |
|---|---|---|---|---|---|---|
| Ingredient | % (w/w) | tablet | (w/w) | tablet | (w/w) | tablet |
| Compound A | 37.5 | 300.0 | 36 | 200.2 | 30 | 150 |
| HydroxyPropyl Cellulose | 4.0 | 32.0 | 5 | 27.8 | 4 | 20 |
| (HPC-Klucel EXF) | | | | | | |
| Methocel K100 LV | 20.0 | 160.0 | 25 | 139.0 | 25 | 125 |
| Mannitol (Mannogem XL SD) | 18.8 | 150.0 | 16.5 | 91.7 | 18.65 | 93.25 |
| Dicalcium Phosphate | 18.8 | 150.0 | 16.5 | 91.7 | 21.35 | 106.75 |
| (Emcompress) | | | | | | |
| Magnesium stearate | 1.0 | 8.0 | 1 | 5.6 | 1 | 5 |
| Total | 100 | 800.0 | 100 | 556.0 | 100 | 500 |

TABLE 5C

| | | mg/ | % | mg/ | % | mg/ |
|---|---|---|---|---|---|---|
| Ingredient | % (w/w) | tablet | (w/w) | tablet | (w/w) | tablet |
| Compound A | 37.5 | 300.0 | 36 | 200.2 | 30 | 150 |
| HydroxyPropyl Cellulose | 4.0 | 32.0 | 5 | 27.8 | 4 | 20 |
| (HPC-Klucel EXF) | | | | | | |
| Carbopol 71G | 20.0 | 160.0 | 25 | 139.0 | 25 | 125 |
| Mannitol (Mannogem XL SD) | 18.8 | 150.0 | 16.5 | 91.7 | 18.65 | 93.25 |
| Dicalcium Phosphate | 18.8 | 150.0 | 16.5 | 91.7 | 21.35 | 106.75 |
| (Emcompress) | | | | | | |
| Magnesium stearate | 1.0 | 8.0 | 1 | 5.6 | 1 | 5 |
| Total | 100 | 800.0 | 100 | 556.0 | 100 | 500 |

Suppliers and grades for various components of the tablet are shown in Table 6.

TABLE 6

| Component | Manufacturer | Supplier Grade |
|---|---|---|
| PolyOx | DuPont | N60K |
| Methocel K100 LV | DuPont | Methocel K4M |
| | | Premium CR |
| HydroxyPropyl Cellulose (HPC) | Ashland | Klucel EXF |
| Dicalcium Phosphate | JRS Pharma | Emcompress |
| Mannitol | SPI Pharma | Mannogem XL SD |
| Carbopol | Lubrizol | 71G |
| Magnesium stearate | Mallinckrodt | Hyqual 5712 |
| | | (vegetable source) |

### Example 3. Preparation of Modified-Release Tablets

An exemplary process for the preparation of modified-release tablets is as follows.

Granulation was prepared as follows. Mannitol, Compound A, Hydroxypropyl cellulose (Kluel EXF), and release controlling polymer (PolyOx, Methocel, or Carbopol) is weighed out and screened through a 20 mesh. Screened powder is added to the appropriately sized blender. The mixture was blended for 15 minutes. Magnesium stearate was screened through a #30 mesh screen and added to the blender. The mixture was blended for 3 minutes.

The final blend was compressed using appropriately sized tooling to make sustained release tablet of Compound A. The block flow diagram of the processing steps is shown in Scheme 1.

Examples of preparations of modified-release tablets are as follows:

(a) Example 3-1: A 0.5 kg blend of Compound A 300 mg with 37.5% PolyOx N60K was prepared by direct blending and compressing. Eighty tablets are compressed at 800-mg target tablet size and 15-kP target hardness and the release profile is determined. The release rate of the tablets is determined compared to the immediate-release tablet.

(b) Example 3-2: A 0.5 kg batch of Compound A powder blend with 20% Methocel K100LV by direct blending and compression was prepared. Eighty tablets are compressed at 800-mg target tablet size and 15-kP hardness are compressed and the release profile is determined. The release rate of the tablets is compared to the modified-release capsule at time points <6 hrs and at time points ≥6 hrs.

(c) Example 3-3: A 0.5 kg batch of Compound A powder blend with 20% Carbopol 71G by blending and compression was prepared. Eighty tablets are compressed at 800-mg target tablet size and 15-kP hardness are compressed and the release profile is determined. The release rate of the tablets is compared to the modified-release capsule.

(d) Each of the three blends were compressed using 0.3071″×0.7087″ oblong shaped tooling.

### Example 4: Preparation of Tablets Containing Modified Release Compositions Containing Eudrigit®

An example of a modified-release tablets containing Eudrigit® is shown in Table 7.

TABLE 7

| Material | mg/tab | w/w % |
|---|---|---|
| Compound A | 300 | 39.0 |
| Eudragit ® RLPO | 100 | 13.0 |
| HPC (Klucel ® EXF) | 55 | 7.1 |
| Mannitol | 50 | 6.5 |
| Dicalcium Phosphate | 100 | 13.0 |
| (Emcompress ®) NF | | |
| Magnesium Stearate NF | 9 | 1.2 |
| Total | 614 | 100.0 |

### Example 5: Preparation of Tablets Containing Modified Release Compositions Containing Ethocel™

An example of a modified-release tablets containing Ethocel™ is shown in Table 8.

TABLE 8

| Material | mg/tab | w/w % |
|---|---|---|
| Compound A | 300 | 39.0 |
| Ethocel ™ 10 cp | 100 | 13.0 |
| HPC (Klucel ® EXF) | 55 | 7.1 |
| Mannitol | 50 | 6.5 |
| Dicalcium Phosphate | 100 | 13.0 |
| (Emcompress ®) NF | | |
| Magnesium Stearate NF | 9 | 1.2 |
| Total | 614 | 100.0 |

### Example 6: Preparation of Tablets Containing Modified Release Compositions Containing Carbopol®

An example of a modified-release tablets containing Carbopol® is shown in Table 9.

TABLE 9

| Material | mg/tab | w/w % |
|---|---|---|
| Compound A | 300 | 39.6 |
| Carbopol ® 71G NF | 110 | 14.5 |
| HPC (Klucel ® EXF) | 57 | 7.5 |
| Mannitol | 120 | 15.9 |
| Dicalcium Phosphate | 150 | 19.8 |
| (Emcompress ®) NF | | |
| Magnesium Stearate NF | 10 | 1.3 |
| Total | 747 | 100.0 |

### Example 7: Preparation of Tablets Containing Modified Release Compositions Containing HPC (HXF Grade)

An example of a modified-release tablets containing HPC (HXF grade) is shown in Table 10.

TABLE 10

| Material | mg/tab | w/w % |
|---|---|---|
| Compound A | 300 | 39.6 |
| HPC (Klucel ™ HXF) | 120 | 15.9 |
| HPC (Klucel ™ EXF) | 57 | 7.5 |
| Mannitol | 120 | 15.9 |
| Dicalcium Phosphate | 150 | 19.8 |
| (Emcompress ®) NF | | |
| Magnesium Stearate NF | 10 | 1.3 |
| Total | 757 | 100.0 |

### Example 8: Preparation of Tablets Containing Modified Release Compositions Containing HPMC (Methocel™)

An example of a modified-release tablets containing HPMC (Methocel™) is shown in Table 11.

TABLE 11

| Material | mg/tab | w/w % |
|---|---|---|
| Compound A | 300 | 39.0 |
| Methocel ™ K100M | 125 | 16.2 |
| HPC (Klucel ™ EXF) | 55 | 7.1 |
| Mannitol | 160 | 20.8 |
| Dicalcium Phosphate | 120 | 15.6 |
| (Emcompress ®) NF | | |
| Magnesium Stearate NF | 10 | 1.3 |
| Total | 770 | 100.0 |

### Example 9: Preparation of Tablets Containing Modified Release Compositions Containing Carbopol® and Ethocel™

In some tablets, the hydrophilic and hydrophobic polymers are used in conjunction in order to carefully control drug release.

An example of a modified-release tablets containing Carbopol and Ethocel™ is shown in Table 12.

TABLE 12

| Material | mg/tab | w/w % |
|---|---|---|
| Compound A | 300 | 39.6 |
| Carbopol ® 71G NF | 70 | 9.2 |
| Ethocel ™ | 60 | 7.9 |
| HPC (Klucel ™ EXF) | 67 | 8.9 |
| Mannitol | 220 | 29.1 |
| Magnesium Stearate NF | 10 | 1.3 |
| Total | 727 | 100.0 |

### Example 10: Preparation of Tablets Containing an Immediate Release Tablet Core Coated with Controlled Release Coating

Polyvinyl acetate (PVAc, MW 450,000) is a hydrophobic polymer. PVAc is insoluble and does not strongly swell as other extended release polymers such as xanthan gum, guar gum or locust bean gum, and hydroxyalkylated or carboxyalkylated cellulosic excipients. PVAc is available as 30% dispersion comprising of 2.7% povidone K30 as a pore former and 0.3% sodium lauryl sulfate (SLS) as a stabilizer/wetting agent. The povidone plays an important role in releasing the drug molecules from insoluble PVAc films and SLS provides an advantage for spreading the polymer during coating, hence leading to homogeneous films. In addition, the self-sealing property of PVAc is also crucial to prevent instant release and avoid any dose dumping (Ensslin et al., 2009).

An example of an immediate release tablet core coated with controlled release coating is shown in Table 13.

TABLE 13

| Material | mg/tab | w/w % |
|---|---|---|
| Compound A | 300 | 42.4 |
| HPC (Klucel ™ HXF) | 75 | 10.6 |
| Microcrystalline Cellulose | 300 | 42.4 |
| Magnesium Stearate NF | 10 | 1.4 |
| Core Sub-Total | 685 | 96.9 |
| PVAc Coating (2.7% povidone | 22 | 3.1 |
| K30 and 0.3% SLS)* | | |
| Total Coated Tablet Weight | 707 | 100 |

*sprayed as 30% (w/w dispersion)

### Example 11: Preparation of Tablets Containing a Hydrophilic Matrix Coated with Controlled Release Coating

An example of a tablet containing a hydrophilic matrix coated with controlled release coating is shown in Table 14

TABLE 14

| Material | mg/tab | w/w % |
|---|---|---|
| Compound A | 300 | 35.7 |
| HPC (Klucel ™ EXF) | 65 | 7.7 |
| Carbopol ® 71G NF | 70 | 8.3 |
| Mannitol | 120 | 14.3 |
| MCC | 250 | 29.8 |
| Magnesium Stearate NF | 10 | 1.2 |
| Core Sub-Total | 815 | 97.0 |
| Ethocel ™ Coating (containing | 25 | 3.0 |
| 15% HPC pore former) | | |
| Total | 840 | 100.0 |

### Example 12: In Vitro Dissolution

The rate of release of the pharmaceutical formulation is described according to standardized dissolution testing procedures as found in the U.S. Pharmacopoeia, where less than 50% of the drug is released within 1 hour of measurement and not less than 70% of the drug is released at the targeted dosing period, such as an 8 to at least 12 hour period.

In vitro drug release is performed using a USP apparatus II (paddle), with 500 mL of dissolution medium maintained at 37±1° C. for 12 h, at 50 rpm. 0.1N HCl (pH 1.2) is used as the dissolution medium for the first 2 h followed by pH 7.2 phosphate buffer for the next 10 h. Samples are withdrawn at 0.5, 2, 4, 8, 12, 18, and 24 h intervals, respectively. The amounts of dissolved drug are then determined spectrophotometrically (UV/Vis), using filtered portions of the samples. The drug released at any time interval is obtained by calculating the mean cumulative percent of drug release belonging to six tablets from each formulation.

### Example 13: Preparation and Characterization of 300 mg Strength Tablets Containing a Hydrophilic Polymer Matrix

### Tablet Preparation

Three trials were prepared as per the formulations provided in Table 15 using the general manufacturing procedure described in Scheme 1 at a scale of 500 g each. Briefly, the manufacturing process involves a simple direct blending and compression process.

As described above in Example 3, Example 13-1 was prepared using 25% PolyOx™ N60 K; Example 13-2 was prepared using 20% Methocel™ K100 LV; and Example 13-3 was prepared using 20% Carbopol® 71G.

TABLE 15

| | Example 13-1 | | Example 13-2 | | Example 13-3 | |
|---|---|---|---|---|---|---|
| Ingredient | mg/tab | w/w % | mg/tab | w/w % | mg/tab | w/w % |
| Compound A | 300.0 | 37.5 | 300.0 | 37.5 | 300.0 | 37.5 |
| HPC (Klucel ™ EXF) | 32.0 | 4.0 | 32.0 | 4.0 | 32.0 | 4.0 |
| PolyOx ™ N60K | 200.0 | 25.0 | | | | |
| Methocel ™ K100 LV | | | 160.0 | 20.0 | | |
| Carbopol ® 71G | | | | | 160.0 | 20.0 |
| Mannitol | 130.0 | 16.3 | 150.0 | 18.8 | 150.0 | 18.8 |
| Dicalcium Phosphate | 130.0 | 16.3 | 150.0 | 18.8 | 150.0 | 18.8 |
| Magnesium Stearate | 8.0 | 1.0 | 8.0 | 1.0 | 8.0 | 1.0 |
| Totals: | 800.0 | 100.0 | 800.0 | 100.0 | 800.0 | 100.0 |

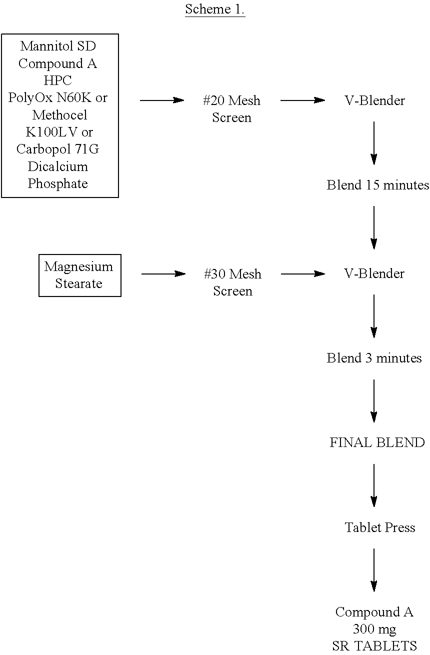

### Physical Characterization

Compound A represents 37.5% of the formulation and since the manufacturing process involves a simple direct blending and compression process, the physical characteristics of the starting Compound A have a significant influence of the final blend itself. Compound A has a relatively coarse particle size distribution and mediocre flow characteristics. After blending with excipients, the flow characteristics generally improved for Examples 13-1 (PolyOx N6K) and Example 13-3 (Carbopol 71G). [Unlike PolyOx N6K and Carbopol 71G which are granular and free-flowing, Methocel K100 LV is a fine powder with generally poor flow characteristics.] However, in spite of the poorer flow characteristics of Example 13-2 (Methocel K100 LV), all formulations compressed well during tableting.

A summary of the physical tablet characteristics for the three formulations are provided in Tables 16 and 17.

TABLE 16
Particle Size Distribution

| | | Example 13-1 | Example 13-2 | Example 13-3 |
|---|---|---|---|---|
| | | 25% | 20% | 20% |
| | | PolyOx ™ | Methocel ™ | Carbopol ® |
| Tests | Compound A | N60K | K100 LV | 71G |
| Bulk Density | 0.431 g/mL | 0.467 g/mL | 0.451 g/mL | 0.467 g/mL |
| Tapped Density | 0.758 g/mL | 0.633 g/mL | 0.758 g/mL | 0.658 g/mL |
| Carr’s Index | 43.14% | 26.22% | 40.50% | 29.03% |
| Hausner Ratio | 1.76 | 1.36 | 1.68 | 1.41 |
| US Mesh Size | % Retained | | | |
| 20 | 6.4 | 0.0 | 0.0 | 0.0 |
| 30 | 19.2 | 6.0 | 6.8 | 6.0 |
| 40 | 14.6 | 7.6 | 7.2 | 6.8 |
| 60 | 35.8 | 17.6 | 14.8 | 26.2 |
| 80 | 12.8 | 19.6 | 20.4 | 20.8 |
| 100 | 4.8 | 14.4 | 9.6 | 14.0 |
| 120 | 2.0 | 6.8 | 6.0 | 4.8 |
| 170 | 4.0 | 15.6 | 15.6 | 14.8 |
| 200 | 1.0 | 6.8 | 9.6 | 4.0 |
| 230 | 0.0 | 1.6 | 2.4 | 0.8 |
| Pan | 0.0 | 4.0 | 6.0 | 1.2 |

TABLE 17
Physical Tablet Characteristics

| Example 13-1 | Example 13-2 | Example 13-3 |
|---|---|---|
| 25% | 20% | 20% |
| PolyOx ™ | Methocel ™ | Carbopol ® |
| N60K | K100 LV | 71G |
| Appearance | | |
| White to off | White to off | White to off |
| white, plain, | white, plain, | white, plain, |
| biconvex, | biconvex, | biconvex, |
| 0.3071″ × | 0.3071″ × | 0.3071″ × |
| 0.7087″ caplet | 0.7087″ caplet | 0.7087″ caplet |

Weight (mg)

| | | | |
|---|---|---|---|
| Max | 805.9 | 809.8 | 810.9 |
| Min | 796.2 | 784.3 | 792.3 |
| Avg | 801.20 | 797.23 | 800.27 |
| (10 tablets) | | | |
| % RSD | 0.41 | 1.2 | 0.73 |

Thickness (mm)

| | | | |
|---|---|---|---|
| Max | 6.53 | 6.76 | 6.64 |
| Min | 6.50 | 6.62 | 6.60 |
| Avg | 6.51 | 6.69 | 6.62 |

Hardness (kp)

| | | | |
|---|---|---|---|
| Max | 16.0 | 17.0 | 17.1 |
| Min | 14.2 | 11.3 | 14.4 |
| Avg | 14.97 | 13.81 | 15.44 |

Friability

| | | | |
|---|---|---|---|
| % Friability | 0.11 | 0.22 | 0.12 |

A target tablet hardness of approximately 15 kp was used for each of the trials. No sticking or picking issues were encountered and the resulting tablets were free of defects. Tablet friability results were very good.

No sticking or punch filming was detected with any of the three formulations. Good flow and uniform weights and hardness were observed for Examples 13-1 (PolyOx™ N60K) and Example 13-3 (Carpopol® 71G). Fair flow with some weight fluctuation and variable hardness was observed for Example 13-2 (Methocel™ K100 LV).

### Dissolution Testing

Samples of each of the formulations were submitted for dissolution testing.

The dissolution results from the three formulations determined by USP apparatus 1 (baskets) produced dissolution profiles that were much slower than anticipated, particularly for Example 13-3 containing Carbopol® 71G releasing only 33% of the drug within 18 hrs. The Example 13-2 containing Methocel™ K100LV produced a relatively linear profile releasing approximately 60% of drug within 18 hrs. Although the Example 13-1 containing PolyOx™ N60K also released 60% of drug within 18 hrs, this polymer had a significantly high degree of variability (% RSD) at all time points tested while the other two polymers produced much more consistent results.

However, baskets may not provide enough hydrodynamic interaction with the matrix tablets and the fine mesh material of the baskets can potentially become obstructed with gel material eroding from the tablets. Therefore, dissolution testing was repeated on tablets containing Carbopol® 71G and Methocel™ K100LV using USP apparatus 2 (paddles) at 75 RPM.

The dissolution results generated using USP apparatus 2 (paddled) in place of apparatus 1 produced a faster release profile for both polymers after 6 hours compared to the initial results. However in the case of the formulation containing 20% Carbopol 71G (Example 13-3), the results dramatically increased at the 18 hour time point suggesting that the tablet matrix may have broken apart due to the increased hydrodynamic forces involved.

A summary of the dissolution results for the three formulations are provided in Table 18.

TABLE 18

| | Formulation | | | | |
|---|---|---|---|---|---|
| | Example 13-1 | Example 13-2 | | Example 13-3 | |
| | 25% | 20% | | 20% | |
| | PolyOx ™ | Methocel ™ | | Carbopol ® | |
| | N60K | K100 LV | | 71G | |
| | USP Apparatus | | | | |
| | Baskets | Baskets | Paddles | Baskets | Paddles |
| Time (hrs) | % Dissolved/(% RSD) | | | | |
| 0 | 0 | 0 | 0 | 0 | 0 |
| 1 | 3.3/(21.6) | 11.6/(5.0) | 12.1/(7.1) | 2.5/(4.4) | 2.3/(3.0) |
| 3 | 8.2/(18.0) | 22.3/(3.4) | 24.5/(6.6) | 6.4/(2.4) | 6.8/(2.3) |
| 6 | 18.2/(17.7) | 34.8/(2.6) | 40.1/(29) | 12.3/(1.6) | 14.8/(2.1) |
| 8 | 25.7/(16.8) | 41.2/(2.6) | 48.9/(1.6) | 16.4/(0.7) | 21.2/(2.4) |
| 12 | 39.9/(15.2) | 50.6/(3.0) | 64.0/(0.4) | 24.2/(1.7) | 38.1/(3.2) |
| 18 | 58.1/(13.6) | 60.3/(3.6) | 80.1/(0.5) | 32.8/(1.7) | 91.1/(5.0) |
| inf. | 61.3 | 63.8 | 83.8 | 34.9 | 98.0 |

### Example 14: Small-Scale Preparation of 300 mg Strength Tablets Containing Low Level Hydrophilic Polymer Matrix

In order to accelerate the dissolution rates observed in Example 13, new tablets were formulated with lower levels of polymer used. Four initial small trial (50 g) blends we prepared according to the formulations in Table 19. With the reduction in polymer level, the level of soluble filler (mannitol) was increased accordingly to keep the net weight of the dosage unchanged. A higher level of soluble filler may further facilitate the rate of release.

TABLE 19

| | Example 14-1 | | Example 14-2 | | Example 14-3 | | Example 14-4 | |
|---|---|---|---|---|---|---|---|---|
| Ingredient | mg/tab | w/w % | mg/tab | w/w % | mg/tab | w/w % | mg/tab | w/w % |
| Compound A | 300.0 | 37.5 | 300.0 | 37.5 | 300.0 | 37.5 | 300.0 | 37.5 |
| HPC (Klucel ™ EXF) | 32.0 | 4.0 | 32.0 | 4.0 | 32.0 | 4.0 | 32.0 | 4.0 |
| Methocel ™ K100 LV | 120.0 | 15.0 | 80.0 | 10.0 | | | | |
| Carbopol ® 71G | | | | | 100.0 | 12.5 | 80.0 | 10.0 |
| Mannitol | 190.0 | 23.8 | 230.0 | 28.8 | 210.0 | 26.3 | 230.0 | 28.8 |
| Dicalcium Phosphate | 150.0 | 18.8 | 150.0 | 18.8 | 150.0 | 18.8 | 150.0 | 18.8 |
| Magnesium Stearate | 8.0 | 1.0 | 8.0 | 1.0 | 8.0 | 1.0 | 8.0 | 1.0 |
| Totals: | 800.0 | 100.0 | 800.0 | 100.0 | 800.0 | 100.0 | 800.0 | 100.0 |

Tablet samples from each of these blends were manually compressed using an MTCM-1 hydraulic press and placed into a dissolution apparatus fitted with vessels containing 900 ml of water using apparatus 2 (paddles) at 75 RPM for physical observation of the tablets over 8 hrs.

The tablets containing 10% levels of polymer showed steady and gradual erosion of the tablet over time. The tablet containing Carbopol® 71G (Example 14-4) actually began to break up into larger pieces at 6 hours and was more fully broken up by 8 hrs.

Based upon these observations, two larger formulations of Compound A tablets were prepared based upon the formulations of Examples 14-2 (10% Methocel™ K100 LV) and 14-4 (10% Carbopol® 71G).

### Example 15: Preparation and Characterization of 300 mg Strength Tablets Containing 10% Hydrophilic Polymer Matrix

### Tablet Preparation

Two trials were prepared as per the formulations provided in Table 19 using the general manufacturing procedure described in Scheme 1 (Example 13) at a scale of 550 g each. Briefly, the manufacturing process involves a simple direct blending and compression process.

Example 15-1 was prepared using 10% Carbopol® 71G; and Example 15-2 was prepared using 10% Methocel™ K100 LV.

TABLE 19

| | Example 15-1 | | Example 15-2 | |
|---|---|---|---|---|
| Ingredient | mg/tab | w/w % | mg/tab | w/w % |
| Compound A | 300.0 | 37.5 | 300.0 | 37.5 |
| HPC (Klucel ™ EXF) | 32.0 | 4.0 | 32.0 | 4.0 |
| Methocel ™ K100 LV | | | 80.0 | 10.0 |
| Carbopol ® 71G | 80.0 | 10.0 | | |
| Mannitol | 230.0 | 28.8 | 230.0 | 28.8 |
| Dicalcium Phosphate | 150.0 | 18.8 | 150.0 | 18.8 |
| Magnesium Stearate | 8.0 | 1.0 | 8.0 | 1.0 |
| Totals: | 800.0 | 100.0 | 800.0 | 100.0 |

### Physical Characterization

The flow characteristics for Example 15-2 (Eap Methocel™ K100 LV) were significantly better than Example 13-2 (201 Methocel™ K100 LV). However, the Carbopol® 71G example (Example 15-1) still maintained better flow characteristics and a relatively coarser particle size distribution at this lower polymer level.

Both examples were successfully compressed on the Manesty Betapress at a speed of42 rpm.

A summary of the physical tablet characteristics for the three formulations are provided in Tables 20 and 21.

TABLE 20
Particle Size Distribution

| | | Example 15-1 | Example 15-2 |
|---|---|---|---|
| | | 10% | 10% |
| | | Carbopol ® | Methocel ™ |
| Tests | Compound A | 71G | K100 LV |
| Bulk Density | 0.431 g/mL | 0.500 g/mL | 0.485 g/mL |
| Tapped Density | 0.758 g/mL | 0.658 g/mL | 0.677 g/mL |
| Carr’s Index | 43.14% | 24.01% | 28.36% |
| Hausner Ratio | 1.76 | 1.32 | 1.40 |
| US Mesh Size | % Retained | | |
| 20 | 6.4 | 0.4 | 0.8 |
| 30 | 19.2 | 15.2 | 8.4 |
| 40 | 14.6 | 9.6 | 7.2 |
| 60 | 35.8 | 28.4 | 11.4 |
| 80 | 12.8 | 20.8 | 15.2 |
| 100 | 4.8 | 7.6 | 14.4 |
| 120 | 2.0 | 3.6 | 8.0 |
| 170 | 4.0 | 8.8 | 16.0 |
| 200 | 1.0 | 3.2 | 8.0 |
| 230 | 0.0 | 0.0 | 2.4 |
| Pan | 0.0 | 1.6 | 6.8 |

TABLE 21
Physical Tablet Characteristics

| Example 15-1 | Example 15-2 |
|---|---|
| 10% | 10% |
| Carbopol ® | Methocel ™ |
| 71G | K100 LV |
| Appearance | |
| White to off | White to off |
| white, plain, | white, plain, |
| biconvex, | biconvex, |
| 0.3071″ × | 0.3071″ × |
| 0.7087″ caplet | 0.7087″ caplet |

Weight (mg)

| | | |
|---|---|---|
| Max | 803.9 | 807.1 |
| Min | 795.6 | 784.5 |
| Avg | 799.38 | 794.10 |
| (10 tablets) | | |
| % RSD | 0.34 | 0.84 |

Thickness (mm)

| | | |
|---|---|---|
| Max | 6.47 | 6.52 |
| Min | 6.44 | 6.49 |
| Avg | 6.45 | 6.50 |

Hardness (kp)

| | | |
|---|---|---|
| Max | 15.9 | 16.0 |
| Min | 14.0 | 12.0 |
| Avg | 15.04 | 13.81 |

Friability

| | | |
|---|---|---|
| % Friability | 0.14 | 0.20 |

A target tablet hardness of approximately 15 kp was used for each of the trials. No sticking or picking issues were encountered and the resulting tablets were free of defects. Tablet friability results were very good.

No sticking or punch filming was detected with either formulation. Good flow and uniform weight and hardness was observed for Example 15-1 (Carpopol® 71G). Fair flow with some weight fluctuation and variable hardness was observed for Example 15-2 (Methocel™ K100 LV).

### Dissolution Testing

Samples of each of the formulations were submitted for dissolution testing using USP apparatus 2 (paddles) as described in Example 13.

Example 15-2 (10% Methocel™ K100 LV) shows a dissolution profile that sustains the release of Compound A for up to 18 hrs when 95.4% of the drug has been released. This is significantly faster than the previous trial containing 20% Methocel™ K100 LV (Example 13-2).

In contrast, the dissolution profile of Example 15-1 (10% Carbopol® 71G) showed only a modest increase in the rate of release over the early time points when compared to the trial containing 20% Carbopol® 71G (Example 13-3). As with Example 13-3, the release increased dramatically at the 12 hour time point, indicating that the matrix may have broken up.

A summary of the dissolution results for the two formulations are provided in Table 22.

TABLE 22

| | Formulation | |
|---|---|---|
| | Example 15-1 | Example 15-1 |
| | 10% | 10% |
| | Carbopol ® | Methocel ™ |
| | 71G | K100 LV |
| | USP Apparatus | |
| | Paddles | Paddles |
| Time (hrs) | % Dissolved/(% RSD) | |
| 0 | 0 | 0 |
| 1 | 3.8/(1.9) | 33.4/(8.4) |
| 3 | 10.2/(0.6) | 57.5/(6.4) |
| 6 | 18.9/(2.0) | 71.9/(4.1) |
| 8 | 24.6/(2.3) | 78.3/(3.3) |
| 12 | 47.5/(14.8) | 87.4/(2.1) |
| 18 | 83.8/(5.9) | 95.4/(1.0) |
| inf. | 93.7 | 97.1 |

Although the rate of release of Compound A improved at the low polymer concentrations (particularly with Methocel™ K100 LV), a 10% polymer level is typically considered to be quite low to achieve a sustained release period of 18 hrs, so such a result is surprising and unexpected. Upon further investigation into the solubility characteristics of Compound A, the solubility of Compound A is actually quite low at pH 7 (1.04 mg/ml after 24 hrs) which is close to that of the dissolution media used for this study (pH 6.8 potassium phosphate buffer). Due to its low solubility at this pH the Compound A matrix tablet release would be limited primarily to the erosion of the gel matrix instead of by both diffusion and erosion. Secondly, the desired sink conditions for the 300 mg strength of Compound A in 900 ml of buffer would be marginal, at best. Additionally, Compound A may be interacting synergistically with the hydrophilic polymers resulting in a stronger matrix than would be typically achieved with the levels used for these specific polymers.

Combined, these results suggest that Methocel™ K100 LV produces a more desirable dissolution profile for Compound A.

### CONCLUSIONS

A sustained release tablet formulation containing 300 mg of compound A is viable. A very low level of Methocel™ K100 LV (10%) produced a sustained release profile of 18 hours and of the three hydrophilic polymers investigated in this study, Methocel™ K100 LV produced the most desirable sustained release dosage profile. Additionally, the use of paddle apparatus (vs basket apparatus) is preferred for evaluation of sustained release matrix based tables.

### Example 16: Clinical Trial—Pharmacokinetics Comparison Study

The objectives of single- and multiple-dose relative bioavailability studies study are to evaluate the PK of the Extended Release formulation and to demonstrate equivalence exposure and other PK parameter (e.g., Cₘᵢₙ) following single- and multiple dose administration of Compound A.

A randomized, open-label, 2-way crossover study is employed. A group of subjects is split, and half are exposed to the compound given twice a day (BID dosing) and half are given the compound in the ER formulation, usually at twice the amount given via a single dose to the patients that received it BID.

Additionally, a 2-period study design is optionally employed where each period is comprised of a single-dose phase which is followed by a multiple-dose phase using the same formulation. Subjects are randomized to receive the ER or immediate release (IR) tablets. Subjects are then switch to the other group in a crossover fashion.

For the ER treatment, a single dose of the ER formulation (e.g., a 600 mg ER tablet) on day 1 is followed by QD dosing of the ER formulation tablets from days 3 through 7. The IR treatment consisted of 2 doses of Compound A in IR 300-mg tablets administered approximately 12 hours apart on day 1 of the study, followed by BID dosing of the IR formulation tablets (12 hours apart) on days 3 through 7. The multiple-dose phase of this study is conducted long enough across both (ER and IR) treatments such that plasma values to reach steady state (˜5 days for IR Compound A). Prior to crossover, a washout period of 72 hours is observed.

During the single-dose phase (day 1) of the study, samples are collected from the subjects that received the ER formulation at predose (0 hours) and 0.5, 1, 2, 3, 4, 6, 9, 12, 24, 36, and 48 hours postdose. For the IR treatment, blood samples are also collected at 0.5, 1, 2, 3, 4, 6, and 9 hours following the evening dose.

During the multiple-dose phase of study, blood samples are collected in a similar fashion to that used in the single dose study at predose through 24 hours postdose. To establish steady state and Cₘᵢₙ values, predose blood samples were collected on the morning of days 3, 4, and 5 of the multiple-dose phase.

The analysis of Compound A is done using typical plasma processing and analytical methods (LC/MS-MS) developed specifically for the compound. Analysis of the PK behavior of Compound A is completed using software developed for that purpose (e.g., WinNonlin, Certara USA, Inc., Princeton, N.J., USA).

### Example 17: Clinical Trial—Food Effect Study

Clinical tests of single dose of the IR formulations of Compound A demonstrated no significant effect of food on the pharmacokinetics of Compound A. To confirm this is the true for the ER formulation, a food effect study is performed.

To test for a food effect, a randomized, open-label, single-dose, 2-period, 2-way crossover study is employed. Subjects (typically 15-25) are randomized to receive Compound A as a tablet under fasted or fed conditions.

For the fasted treatment, subjects receive a single ER tablet (e.g., 600 mg) with water (˜ 0.25 L) following an overnight fast.

The fed treatment consisted of a standard (US Food and Drug Administration) high-fat breakfast 30 minutes prior to administration of a single ER tablet of Compound A (600 mg) with 0.25 L of water. The breakfast used is a high calorie (800-1000 calories), high-fat test meal (50% of total calories) with approximately 150, 350, and 500-600 calories from protein, carbohydrate, and fat, respectively. Subjects are asked to consume breakfast within 30 minutes.

Following drug administration, no additional food is given for approximately four hours post-dose, and additional water is withheld for 2 hours predose and postdose. The washout period for crossover period is typically 72 hours.

PK samples are collected at multiple intervals: predose (0 hours) and 0.5, 1, 2, 3, 4, 6, 9, 12, 24, 36, and 48 hours postdose for each treatment condition.

The analysis of Compound A is done using typical plasma processing and analytical methods (LC/MS-MS) developed specifically for the compound. Analysis of the PK behavior of Compound A is completed using software developed for that purpose (e.g., WinNonlin, Certara USA, Inc., Princeton, N.J., USA).

Those skilled in the art will recognize that various modifications, additions, and substitutions to the illustrative examples set forth herein can be made without departing from the spirit of the invention and are, therefore, considered within the scope of the invention.

What is claimed is:

1. A method of treating a hematological malignancy in an individual in need thereof, comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof.

2. The method of claim 1, wherein the method further comprises administration of a BTK inhibitor.

3. The method of claim 2, wherein the BTK inhibitor is ibrutinib.

4. A method of treating a DLBCL in an individual in need thereof, comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof.

5. The method of claim 4, wherein the DLBCL is ABC-DLBCL.

6. The method of claim 4 or 5, wherein the method further comprises administration of a BTK inhibitor.

7. The method of claim 6, wherein the BTK inhibitor is ibrutinib.

8. A method of treating an AML in a subject in need thereof comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof.

9. The method of claim 8, wherein the method further comprises administration of a BLC2 inhibitor.

10. A method of treating leukemia in a subject in need thereof comprising administering to the individual an extended release composition comprising 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof, wherein the leukemia is chosen from acute lymphoblastic leukemia (ALL), acute myelogenous leukemia (AML), chronic myelogenous leukemia (CML), small lymphocytic lymphoma (SLL), or chronic lymphoblastic leukemia (CLL).

11. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof, wherein the plasma Cmax is no greater than 4,000 ng/mL and the plasma Cmin is no less than 800 ng/mL for the entire 24 hour period between oral dosage form administration.

12. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof wherein the plasma Cmax is no greater than 3,000 ng/mL and the plasma Cmin is no less than 800 ng/mL for the entire 24 hour period between oral dosage form administration.

13. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof wherein the plasma Cmax is no greater than 2,000 ng/mL and the plasma Cmin is no less than 800 ng/mL for the entire 24 hour period between oral dosage form administration.

14. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof wherein the Cmin is no less than 1,000 ng/mL for the entire 24 hour period between oral dosage form administration.

15. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof wherein the Cmin is no less than 900 ng/mL for the entire 24 hour period between oral dosage form administration.

16. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof wherein the Cmin is no less than 800 ng/mL for the entire 24 hour period between oral dosage form administration.

17. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof wherein the Cmin is no less than 700 ng/mL for the entire 24 hour period between oral dosage form administration.

18. A method of treating a disease or disorder mediated by PKCβ signaling in an individual in need thereof, comprising orally administering once per day 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof wherein the Cmin is no less than 600 ng/mL for the entire 24 period between oral dosage form administration.

19. A method of treating a disease or disorder mediated by PKCβ signaling in an individual in need thereof, comprising orally administering once per day a PKCβ inhibitor, or a pharmaceutically acceptable salt thereof wherein inhibition of PKCβ signaling occurs for the entire 24 hour period between oral dosage form administration.

20. The method of claim 19, wherein the PKCβ inhibitor is 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine, or a pharmaceutically acceptable salt thereof.

21. The method of any one of claims 11-20, wherein the disease or disorder mediated by PKCβ signaling is an autoimmune disease or disorder, or cancer.

22. The method of claim 21, wherein the cancer is a hematological malignancy.

23. An extended release pharmaceutical formulation comprising:
a. from about 5% to about 70% of 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine by weight; and
b. a release controlling polymer system comprising:
i. a hydrophilic release controlling polymer;
ii. a hydrophobic release controlling polymer; or
iii. a combination thereof.

24. The extended release pharmaceutical formulation of claim 23, wherein the hydrophilic release controlling polymer is hydroxypropyl methyl cellulose (HPMC), hydroxypropylcellulose (HPC), Poly(ethylene) Oxide, soluble polyvinylpyrrolidone (Povidon), cross-linked polyacrylic acid polymer (Carbopol), or a combination thereof.

25. The extended release pharmaceutical formulation of claim 23, wherein the hydrophilic release controlling polymer is hydroxypropyl methyl cellulose (HPMC), hydroxypropylcellulose (HPC), Poly(ethylene) Oxide, soluble polyvinylpyrrolidone (Povidon), cross-linked polyacrylic acid polymer (Carbopol), or a combination thereof.

26. The extended release pharmaceutical formulation of claim 23, wherein the hydrophilic release controlling polymer is Methocel™ K100 LV, HPC (Klucel™ EXF), PolyOx™ N60K, Carbopol® 71G, or a combination thereof.

27. The extended release pharmaceutical formulation of claim 23, wherein the hydrophobic release controlling polymer is ethylcellulose, hypromellose acetate succinate, cellulose acetate, cellulose acetate propionate, Eudogrit®, natural wax, or a combination thereof.

28. The extended release pharmaceutical formulation of any one of claims 23-27, comprising from 10% to 50% of 5-{1[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine by weight.

29. The extended release pharmaceutical formulation of any one of claims 23-28, comprising from 35% to 45% of 5-{[(2S,5R)-2,5-dimethyl-4-(tetrahydro-2H-pyran-4-ylmethyl)piperazin-1-yl]carbonyl}-N-(5-fluoro-2-methylpyrimidin-4-yl)-6,6-dimethyl-1,4,5,6-tetrahydropyrrolo[3,4-c]pyrazol-3-amine by weight.
